(19) United States
(12) Patent Application Publication
(10) Pub. No.: US 2022/0056447 A1
(43) Pub. Date: Feb. 24, 2022

(54) REGULATION OF NUCLEOTIDE EXCISION REPAIR (NER) BY MICRORNA FOR TREATMENT OF CANCER

(71) Applicant: Nova Southeastern University, Fort Lauderdale, FL (US)

(72) Inventor: Jean J. Latimer, Fort Lauderdale, FL (US)

(73) Assignee: Nova Southeastern University, Fort Lauderdale, FL (US)

(21) Appl. No.: 17/521,127

(22) Filed: Nov. 8, 2021

Related U.S. Application Data
(63) Continuation-in-part of application No. PCT/US2020/032026, filed on May 8, 2020.
(60) Provisional application No. 62/844,792, filed on May 8, 2019.

(51) Int. Cl. C12N 15/113; A61K 31/713; A61K 33/243; A61K 31/704; A61P 35/00

(52) U.S. Cl. CPC C12N 15/113; A61K 31/713; A61K 33/243; C12N 2320/31; A61P 35/00; C12N 2310/141; A61K 31/704

(57) ABSTRACT

Methods for using microRNA, particularly miRRA, to regulate nucleotide excision repair (NER) for treatment of cancer, particularly drug resistant breast cancer (BC) or late-stage breast cancer. The microRNA will effectively lower NER capacity in breast cancer, allowing for application or reapplication of chemotherapy that will be significantly more effective after pretreatment with the microRNA. Pharmaceutical compositions including microRNA are also provided.

### FIELD OF THE INVENTION

The invention is encompassed within the field of oncology and generally relates to therapeutic modalities for treatment of cancers, particularly to the use of biologic drugs for controlling gene and/or protein expression in breast cancer cells, and most particularly to the use of microRNA compositions for suppressing functional capacity of nucleotide excision repair (NER) in breast cancer cells for treatment of breast cancer (BC).

### BACKGROUND

Genomic instability is a hallmark of cancer. The instant inventor has shown that sporadic stage I breast tumors have an intrinsic loss of nuclear excision repair (NER) function and gene/protein expression compared to normal breast tissues, indicating that the loss of NER is an underlying mechanism of genomic instability and plays a fundamental role in carcinogenesis. The inventor further demonstrated that late-stage breast tumors acquired an increase in NER function relative to stage I breast tumors and non-diseased breast tissues. These findings suggest that NER is subject to dysregulation in breast cancer and can be divided into two phases. The first phase is a loss-of-function phase that occurs early and plays a major role in cancer etiology. The second phase is a gain-of-function phase that happens while the tumor progresses from early to advanced cancer and may be a factor associated with tumor aggressiveness and chemotherapy resistance. This does not imply that all stage I cancers proceed to stage IV, but implies that when they do progress, repair capacity increases.

Molecular analyses of NER gene expression in stage I breast tumors have revealed that 19/20 genes were reduced in expression relative to normal breast epithelial tissues. The instant inventor has shown that the majority of NER genes were overexpressed in a cancer scenario in late-stage breast cancer compared to both stage I tumors and normal breast tissues.

Taken together, these data suggest that there is not one gene acting as a master regulator responsible for the change in NER function between stage I and stage IV disease or in the normal scenario and there is not one gene involved in the tissue specificity seen in NER. In fact, since multiple genes are increased in expression in advanced stage tumor lines, the genes in the NER pathway may be coordinately epigenetically regulated. The instant inventor has studied a possible mechanism of such regulation. Dysregulation of this mechanism might then explain the two aberrant NER phenotypes identified in early and late breast cancer.

NER regulation has rarely been studied in breast cancer. The few NER regulation studies that have been published focus on the transcriptional regulation role of the well-known tumor suppressor gene TP53. The two DNA damage recognition genes in the global genomic NER subpathway XPC and DDB2 have been reported to have TP53 response elements, and their expression was strongly correlated with TP53 protein and gene expression. TP53 expression is induced in response to DNA damage, leading to activation of XPC and DDB2 expression and promotion of DNA damage repair, suggesting that NER function is TP53 dependent in humans. However, this conclusion was challenged by the instant inventor in showing that several mutant-p53 breast cancer cell lines exhibited high NER function when compared to non-diseased breast reduction epithelial tissues and stage I breast tumors. This result indicates that NER function in breast cancer is subject to regulation by additional factors other than TP53 status.

MicroRNAs are small, regulatory non-coding RNAs, 19-21 nucleotides in length, that regulate gene expression epigenetically at the post-transcriptional level. Mature microRNA consists of two strands. One strand is responsible for the functional role of the microRNA, called the “guide” or “leading” strand, and the other is thought to be subjected to degradation with no active role: the “passenger” strand. However, recent reports have shown that the passenger strand can also play a functional role in gene regulation, and that it can interact with target genes in a similar mechanism as the leading strand. MicroRNAs bind to a complementary sequence, usually located in the 3′ untranslated region of target mRNAs, leading to either mRNA degradation or inhibition of protein synthesis. However, emerging evidence has shown that microRNAs can also interact with the 5′ untranslated regions or the open reading frames of their target mRNAs. Additionally, several research groups have demonstrated microRNA regulated gene expression at the transcriptional level through binding to putative binding sites located at gene promoters. More than 2000 microRNAs have been discovered, and they have been predicted to target up to one-third of human coding genes. A single microRNA has the capability to target and regulate multiple genes concurrently, which is a unique feature that makes microRNA an appealing epigenetic mechanism by which multiple NER genes may be co-regulated.

MicroRNA dysregulation has been demonstrated in many types of cancer, including breast cancer, and has been linked to many oncogenic features such as proliferation, invasion, metastasis, angiogenesis, and lack of apoptosis. In addition, microRNAs have been shown to play an important role in DNA repair gene regulation, including NER genes. MicroRNAs-373 and -′744-3p have been shown to target a putative binding site located at the 3′ untranslated region of RAD23B mRNA and significantly reduce RAD23B protein expression in MCF-7 and a series of prostate cancer cell lines. MiR-890 has been demonstrated to interact with the 3′ untranslated region of XPC mRNA and subsequently reduce XPC protein expression in several prostate cancer cell lines. However, these reports did not investigate the impact of such protein reduction on NER function.

Xie et al. has demonstrated that microRNA dysregulation impacted NER function. They showed that the reintroduction of miR-192 in HepG2, which is a well-known hepatocellular carcinoma cell line, significantly reduced NER function by suppressing ERCC3 and ERCC4 gene and protein expression. However, this research group used the host cell reactivation assay to measure the functional impact of miR-192 transfection on NER function. Host cell reactivation measures only transcriptional coupled NER repair, which represents a small percentage (3%) of the total NER repair and might not be a true representative functional assay of NER function.

Several groups, such as Plantamura et al. (Frontiers in Oncology 8:article 352 2018), have shown that microRNAs can sensitize cancer cells to DNA-damaging drugs (chemotherapy). Additionally, Szalat and Gao: Szalat et al. demonstrated that NER inhibition, particularly of XPB (DNA helicase encoded by ERCC3), increases sensitivity to alkylating agents in multiple myeloma (Leukemia 32:111-119 2018). Gao et al. showed that microRNA, particularly miR-145, sensitizes breast cancer to doxorubicin by targeting (inhibiting) the multidrug resistance-associated protein-1(MRP1)(Oncotarget 7(37):59714-59726 2016).

In the studies/research described herein, the mechanistic role of microRNAs in NER function and gene regulation in late-stage breast cancer was investigated. It is suspected that the increased NER function in late-stage tumors is due to an absence or a down-regulation of one or more miRNAs that regulate gene expression and/or protein synthesis of multiple canonical NER genes. This study provides new insights into NER regulation in breast cancer, and potentially might be used as the basis to create new treatment avenues to effectively treat highly proficient-NER aggressive breast tumors that are more likely to resist conventional genotoxic chemotherapy regimens.

### SUMMARY

The instant invention provides a new therapeutic modality for the treatment of cancer, particularly breast cancer (BC), and most particularly late-stage and/or chemo-resistant breast cancer, through regulation of nucleotide excision repair (NER) at the level of both gene and protein expression.

Stage specific and epigenetic control of the 20 NER genes and proteins has been shown in breast cancer and leukemia. There are two possible mechanisms for the differential regulation of these genes: DNA and histone methylation and microRNA regulation. This study examines the possibility of microRNA regulation. An analysis of methylation of these genes in isogenically-matched tissue with high-repairing non-tumor adjacent vs low-repairing stage I tumor showed no evidence of differential methylation that correlated with gene expression. An analysis of isogenically-matched low-repairing non-tumor adjacent tissue and low-repairing tumor also showed no evidence of differential methylation that correlated with gene expression (Manasi Pimply thesis).

MicroRNAs naturally exist in the non-malignant cells of the body, which if applied to tumor/cancer cells, lower the functional capacity of DNA repair (in the tumor/cancer cells) through the regulation of both gene and protein expression of several DNA repair genes. Since DNA repair is a critical engine of drug and radiation resistance in malignant cells, this microRNA therapeutic lowers the potential treatment resistance of the malignant cells and allows for greater efficacy of cancer treatment, particularly in cancer stem cells or advanced stage cancers. Since this microRNA already exists in normal breast cells (as well as heart and liver), its application may incur less morbidity when used for treatment. This microRNA is not detectable or is present at very low levels in advanced tumor cell lines. Additionally, use of the microRNA therapeutic may allow for lower doses of chemotherapeutic drugs to be used more effectively in early-stage disease than conventional doses.

In a most basic aspect, the invention provides a new treatment modality for cancer.

In a general aspect, the invention provides compositions and methods for the treatment of cancer, particularly, but not limited to, breast cancer (BC).

In another general aspect, the invention provides compositions and methods for the treatment of solid cancers.

In another general aspect, the invention provides compositions and methods for the treatment of, but not limited to, late-stage, aggressive, and/or drug resistant cancer, particularly, but not limited to late-stage, aggressive, drug resistant breast cancer, triple negative breast cancer (TNBC), and/or luminal-type breast cancer. These cancers are particularly resistant to chemotherapeutic and/or DNA-damaging drugs and radiation.

In an aspect, the invention provides compositions for treatment of breast cancer, particularly, but not limited to, late-stage breast cancer, including microRNA.

In another aspect, the invention provides pharmaceutical compositions for treatment of cancer, particularly, but not limited to, breast cancer, drug resistant breast cancer, late-stage breast cancer, triple negative breast cancer (TNBC), and luminal-type breast cancer, including a therapeutically effective intravenous or injectable dosage of microRNA in a liquid pharmaceutical carrier. The “liquid pharmaceutical carrier” can be any inactive and non-toxic liquid useful for preparation of intravenous medication. The phrase “therapeutically effective dosage” or “therapeutically effective amount” refers to the amount of a composition required to achieve the desired function; for example, desired regulation of nucleotide excision repair (NER) in malignant cells. Malignant cells are cells characterized by uncontrolled growth. The terms “malignant cells”, “cancer cells,” and “tumor cells” are used interchangeably herein.

A preferred, non-limiting embodiment of the microRNA of the described compositions is miR-145, referred to as miRRA. MiR-145 includes two strands of RNA, a guide strand miR-145-5p and a passenger strand miR-145-3p. The compositions can include the guide strand (miR-145-5p), the passenger strand (miR-145-3p), or both the guide and passenger strands.

In another aspect, the invention provides a method for regulating nucleotide excision repair (NER) function in malignant cells. This method includes steps of providing the microRNA compositions described herein and administering the composition to the malignant cells. The terms “regulating”, and “regulation” refer to control of the expression of the genes and/or proteins of NER, including upregulation, downregulation, and silencing. The preferred, but non-limiting, regulation is suppression and/or inhibition of expression of the genes and/or proteins of NER pathways. The preferred, but non-limiting, targets for regulation are the XPC, XPA, and RPA3 genes and proteins of the NER pathways.

In yet another aspect, the invention provides a method for increasing sensitivity to DNA-damaging drugs in malignant cells. This method includes steps of providing the microRNA compositions described herein and administering the composition to the malignant cells. Carrying out the method lowers the ability of the malignant cells to repair DNA damage caused by the chemotherapeutic, DNA-damaging drugs, thus overcoming drug resistance (of the malignant cells). Preferred, but non-limiting, examples of DNA-damaging drugs are cisplatin, doxorubicin, and Adriamycin.

In yet another aspect, the invention provides a method for treating cancer, particularly, but not limited to, breast cancer, in a subject in thereof by suppressing nucleotide excision repair (NER) function in cancer cells. This method includes steps of providing the microRNA compositions described herein and administering the composition to the subject. The term “suppressing” includes inhibition of both gene and protein expression. The term “subject” refers to any human or animal who will benefit from use of the compositions, methods, and/or treatments described herein. A preferred, but non-limiting subject is a human patient having breast cancer. A similar embodiment of this method includes a further step of administering a DNA-damaging drug to the subject either after administering the composition or concurrently with the composition. Preferred, non-limiting, examples of DNA-damaging drugs are cisplatin and Adriamycin.

Another aspect of the invention provides a method for treating a late-stage breast cancer, a drug-resistant breast cancer, a triple negative breast cancer (TNBC), or a luminal-type breast cancer in a subject in need thereof by inhibiting expression of at least one of XPC and XPA proteins in breast cancer cells. Although breast cancer is a preferred embodiment, this method is contemplated for treatment of any malignant disease/cancer and is not limited to breast cancer. The method includes steps of providing a composition including a therapeutically effective intravenous or injectable dosage of microRNA in a liquid pharmaceutical carrier and administering the composition to the subject. The administration of this microRNA composition inhibits or suppresses expression of XPC and XPA proteins in the breast cancer cells. A preferred, non-limiting embodiment of the microRNA of the composition used in this method is miR-145 (miRRA), particularly the guide strand miR-145-5p. A similar embodiment of this method includes a further step of administering a DNA-damaging drug to the subject either after administering the composition or concurrently with the composition. Preferred, non-limiting, examples of DNA-damaging drugs are cisplatin and Adriamycin.

Yet another aspect of the invention provides a method for treating a late-stage breast cancer, a drug-resistant breast cancer, a triple negative breast cancer (TNBC), or a luminal-type breast cancer in a subject in need thereof by inhibiting expression of RPA3 proteins in breast cancer cells. Although breast cancer is a preferred embodiment, this method is contemplated for treatment of any malignant disease/cancer and is not limited to breast cancer. The method includes steps of providing a composition including a therapeutically effective intravenous or injectable dosage of microRNA in a liquid pharmaceutical carrier and administering the composition to the subject. The administration of this microRNA composition inhibits or suppresses expression of RPA3 proteins in the breast cancer cells. A preferred, non-limiting embodiment of the microRNA of the composition used in this method is miR-145 (miRRA), particularly the passenger strand miR-145-3p. A similar embodiment of this method includes a further step of administering a DNA-damaging drug to the subject either after administering the composition or concurrently with the composition. Preferred, non-limiting, examples of DNA-damaging drugs are cisplatin and Adriamycin.

Other objectives and advantages of this invention will become apparent from the following description, wherein are set forth, by way of example, certain embodiments of this invention.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 shows Table 1, which discloses the clinical and molecular characteristics of culture explants and established cells lines that were selected for microRNA profiling.

FIG. 2 is a dendrogram showing microRNA expression patterns of non-diseased breast tissue, stage I breast tumor tissue, and late-stage breast tumor tissue groups.

FIG. 3 is a graph illustrating the criteria used to select candidate microRNA for experiments.

FIG. 4 is a graph showing the leading strand miR-145-5p expression in the late-stage breast tumor tissue group compared to the stage I breast tumor tissue group using the Nanostring microRNA expression panel.

FIG. 5 is a graph showing the passenger strand miR-145-3p expression in the late-stage breast tumor tissue group compared to the stage I breast tumor tissue group using microRNA RT-PCR.

FIG. 6 is a graph showing the impact of miR-145 on nucleotide excision repair (NER) capacity in MDA-MB-231, MCF-7, and JL BTL-12 breast cancer-derived cell lines.

FIG. 7 is a graph showing the impact of miR-145 on cell proliferation in MDA-MB-231, MCF-7, and JL BTL-12 breast cancer-derived cell lines.

FIG. 8 is a graph showing XPC gene expression regulation by miR-145-5p in MDA-MB-231, MCF-7, and JL BTL-12 breast cancer-derived cell lines.

FIG. 9 is a graph showing XPA gene expression regulation by miR-145-5p in MDA-MB-231, MCF-7, and JL BTL-12 breast cancer-derived cell lines.

FIG. 10 is a graph showing RAD23B gene expression regulation by miR-145-5p in MDA-MB-231, MCF-7, and JL BTL-12 breast cancer-derived cell lines.

FIG. 11 is a graph showing ERCC6 gene expression regulation by miR-145-5p in MDA-MB-231, MCF-7, and JL BTL-12 breast cancer-derived cell lines.

FIG. 12 is a graph showing GTF2H4 gene expression regulation by miR-145-5p in MDA-MB-231, MCF-7, and JL BTL-12 breast cancer-derived cell lines.

FIG. 13 is a graph showing RPA3 gene expression regulation by miR-145-3p in MDA-MB-231, MCF-7, and JL BTL-12 breast cancer-derived cell lines.

FIG. 14 is a graph showing ERCC1 gene expression regulation by both strands miR-145-3p and miR-145-5p in MDA-MB-231, MCF-7, and JL BTL-12 breast cancer-derived cell lines.

FIG. 15A is a representative Western Blot analysis of XPC protein expression regulating by miR-145-5p in MDA-MB-231, MCF-7, and JL BTL-12 breast cancer-derived cell lines.

FIG. 15B is a graph showing the fold change of XPC protein expression in MDA-MB-231, MCF-7, and JL BTL-12 breast cancer-derived cell lines.

FIG. 16A is a representative Western Blot analysis of XPA protein expression regulating by miR-145-5p in MDA-MB-231, MCF-7, and JL BTL-12 breast cancer-derived cell lines.

FIG. 16B is a graph showing the fold change of XPA protein expression in MDA-MB-231, MCF-7, and JL BTL-12 breast cancer-derived cell lines.

FIG. 17A is a representative Western Blot analysis of RPA3 protein expression regulating by miR-145-3p in MDA-MB-231, MCF-7, and JL BTL-12 breast cancer-derived cell lines.

FIG. 17B is a graph showing the fold change of RPA3 protein expression in MDA-MB-231, MCF-7, and JL BTL-12 breast cancer-derived cell lines.

FIG. 18 is a schematic representation of commercially available plasmid pEZX-MT06.

FIG. 19 is a graph showing XPC relative luciferase activity.

FIG. 20 is a graph showing XPA relative luciferase activity.

FIG. 21 is a graph showing nucleotide excision repair (NER) capacity of stage I breast tumor cell cultures.

FIG. 22 is a gene expression microarray showing 19 out of 20 canonical NER genes that are significantly downregulated in three stage I tumors (black bars) compared to three breast reduction epithelium (BRE) samples (white bars).

FIG. 23 is a graph showing that NER function increases as tumor stage progresses from stages I-IV.

FIG. 24 is a graph showing differences in gene expression between stage I breast cancers (JL BTL-8 breast cancer-derived cell line) and late-stage breast cancers (SKBR3, JL BTL-12 (stage III), Cama-1, MCF7, BT-20, and MDA MB231 breast cancer-derived cell lines).

FIG. 25 is a graph showing that RPA3 gene expression was significantly reduced in RPA3 siRNA samples compared to mock and NC samples.

FIG. 26 is a graph showing that silencing of the RPA3 gene in MDA MB 231 cells significantly reduced NER function.

FIG. 27 is a gene expression microarray showing hierarchical clustering.

FIG. 28 is a schematic representation of commercially available plasmid pEZX-MT06 similar to the plasmid shown in FIG. 18.

FIG. 29A is a graph showing XPA relative luciferase activity.

FIG. 29B is a graph showing XPC relative luciferase activity.

FIG. 29C is a graph showing RPA3 relative luciferase activity.

FIG. 30 shows Table 5, which summarizes miRRA-3p and miRRA-5p data.

FIG. 31A is a graph showing cytotoxicity studies using MCF-7 luminal type breast cancer (BC) cell line transfected using lipofectamine with miRRA 3-p vs. scrambled RNA negative control NC (N=4) in the presence of cisplatin.

FIG. 31B is a graph showing cytotoxicity studies using MDA MB231 TNBC cell line transfected using lipofectamine with miRRA 3-p vs. scrambled RNA negative control NC (N=4) in the presence of cisplatin.

FIG. 31C is a graph showing cytotoxicity studies using IL BTL-12 luminal type breast cancer (BC) cell line transfected using lipofectamine with miRRA 3-p vs. scrambled RNA NC (N=3) in the presence of cisplatin.

### DETAILED DESCRIPTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described compositions and methods along with any further application of the principles of the invention as described herein, are contemplated as would normally occur to one skilled in the art to which the invention relates.

The experimental studies described herein are based on the continuing research of the instant inventor.

### Experimental Study: Part 1

Chemotherapy resistance is a central problem in the management of patients with breast cancer, and it is one of the leading causes behind tumor recurrence. Numerous chemotherapeutic agents are genotoxic, inducing DNA damage in cancer cells and destroying their ability to proliferate. The cell response at this point is to repair the damage or to commit suicide (apoptosis). However, cancers have been found with increased DNA repair capacity to remove DNA damaged caused by chemotherapy agents, and they are more likely to survive chemotherapy treatment and continue to grow and spread.

Nucleotide excision repair (NER) has been shown to play a role in remediating DNA damage induced by cyclophosphamide and doxorubicin. In fact, cell lines with mutations in several NER genes have been shown to have higher cyclophosphamide and doxorubicin response compared to wild type cell lines, illustrating that NER function might be a clinical prognostic factor that could predict chemotherapy treatment outcomes. The instant inventor recently published that NER gene expression at diagnosis is prognostic for early vs. late recurrence in recurrent pediatric acute lymphocytic leukemia.

The instant inventor demonstrated that, with increasing stage, breast tumor explants exhibit increasing DNA repair, and this increase might be associated with the chemotherapy resistance that most of the tumors at this stage manifest. The instant inventor's aim at the beginning of this study was to identify a mechanism through which NER is upregulated.

Neither the loss of NER function from the non-diseased state to in the early-stage tumor, nor the gain of function in the late stage was driven by the effect of a single NER gene. Both phenotypes were consequences of dysregulation of multiple genes in the pathway, suggesting that these genes, if they are regulated, might be regulated by a shared mechanism that is most likely epigenetic. RNA sequencing now being performed (in the lab of the instant inventor) has not yet identified mutations in any of the target genes discussed in this application, nor have mutations been seen in the cancer genome atlas than can explain these phenomena. Therefore, microRNAs have emerged as plausible epigenetic NER regulatory elements because of their ability to regulate gene and protein expressions of multiple genes concurrently. However, this does not rule out that other epigenetic mechanisms, such as DNA or histone methylation, are not also present. However, data from Manasi Pimpley's thesis, referred to above, have not supported methylation as the mechanism for differential DNA repair in matching isogenic sets of tissue that included high NER capacity non-tumor adjacent tissue vs. low NER capacity tumor. Similar comparisons were made in a matched low NER capacity non-tumor adjacent explant vs. a low NER capacity tumor. In these pairs, gene expression did not correlate with methylation status in the gene, the CpG islands of the gene, or the 5′ promoter of the gene using methylation arrays.

In recent years, numerous efforts have been made by research groups and pharmaceutical companies worldwide to develop microRNA-based therapies that might be used clinically to treat variety of diseases including cancer. In fact, two microRNAs have already made it to clinical trials, one of which is miR-34, which is proposed to be used to treat several cancer types, including lymphoma, melanoma, myeloma, liver, lung, and renal cancers. This work increases the clinical significance of microRNAs as potential regulatory elements in the NER pathway.

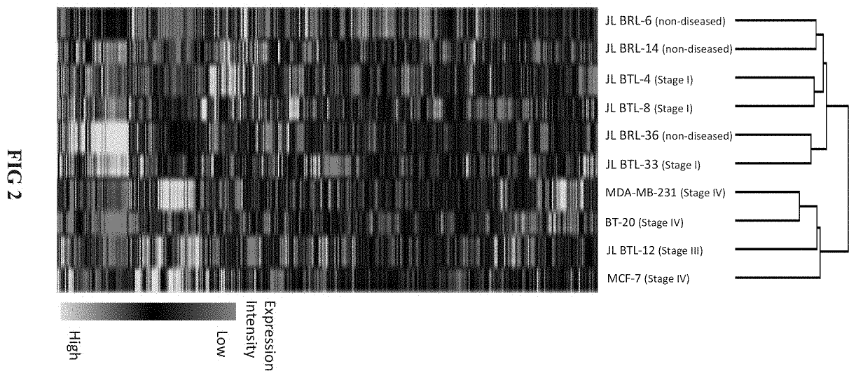

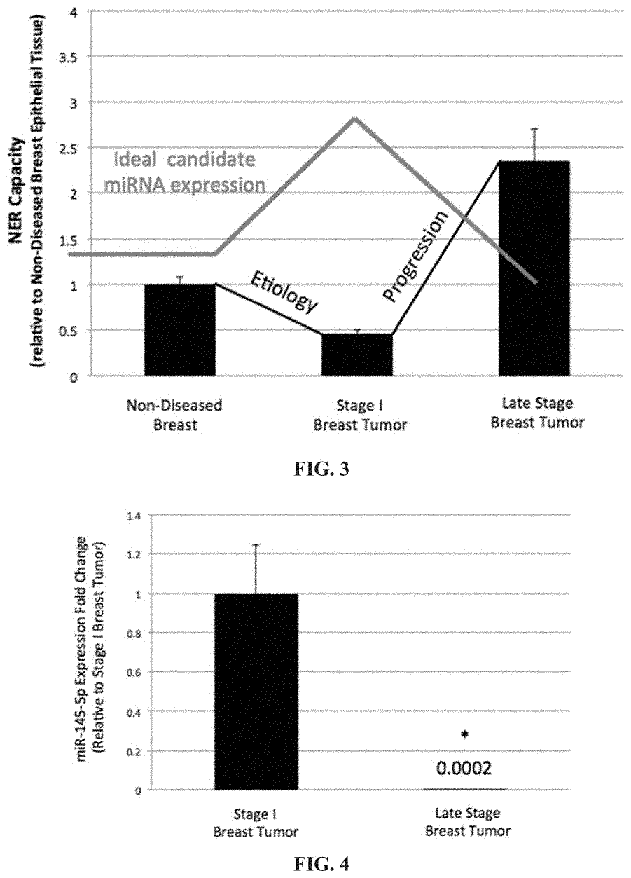

The instant inventor examined expression of 800 microRNAs in a series of non-diseased breast cultures, stage I breast tumor cultures, and late-stage breast tumor explants and cell lines using the Nanostring nCounter Human v3 microRNA expression panel to identify candidate microRNAs that might be involved in NER regulation. The ideal candidate microRNA expression is inversely correlated with NER function in these groups; significantly overexpressed in the stage I tumors compared to non-diseased breast tissues and significantly under-expressed in late-stage breast tumors compared to stage I breast tumors (FIG. 3).

Other microRNA expression profiling platforms, such as microarray, now offers panels of more than 2000 microRNAs for future work. One other possible explanation is that microRNAs might not be significantly involved in breast cancer etiology-related phenomena, particularly the loss of NER function in stage I tumors.

In fact, the late-stage samples had very distinctive microRNA signatures compared to non-diseased tissue and stage I tumors (FIG. 2), illustrating how these advanced tumors are fundamentally different compared to early-stage tumors. This is another piece of evidence that showed that these advanced tumors research models are not representative of early breast tumors. Taken together, the discrepancy of microRNA dysregulation between the etiology and progression phases might suggest that the loss and gain NER of function are occurring by independent mechanisms and that microRNAs might be primarily involved in the gain-of-function phenomenon.

miR-145-5p was the most significantly under-expressed microRNA in late-stage tumors compared to stage I tumors (FIG. 4), which is consistent with several reports that have shown that miR-145-5p was one the most significant downregulated microRNAs in breast cancer. MiR-145 (miRRA) was found to have seeding regions for multiple NER genes using the three microRNA predicted target databases.

Interestingly, the passenger strand miR-145-3p, that was traditionally believed to have no functional effect, was found to have the potential to interact with five NER genes, including RPA3, the most significant overexpressed NER gene in late-stage tumor cell lines compared to primary cultures of stage I breast tumors. This finding suggests that this strand might also be involved in gene regulation of multiple NER genes.

The inventor found that both the leading strand miR-145-5p, and the passenger strand miR-145-3p, had significant effects on NER function in the three advanced stage breast cancer-derived cell lines MDA-MB-231, MCF-7, and IL BTL-12 (FIG. 6), suggesting that both strands contribute to regulate NER function in late breast cancer, and that the loss of miR-145 expression might be a molecular factor behind the increase in NER function in such tumors. Finding an active role for the passenger strand in this study challenges the traditional concept of the non-functionality of the passenger strand and supports a more recent concept of microRNA function from several reports that have shown that the passenger strand was actively involved in regulating gene and protein expression. In fact, a study was published showing that both miR-145 strands, miR-145-5p and miR-145-3p, cooperate to suppress the expression of the oncogene metadherin at both the gene and protein levels in lung squamous cell carcinoma cell lines.

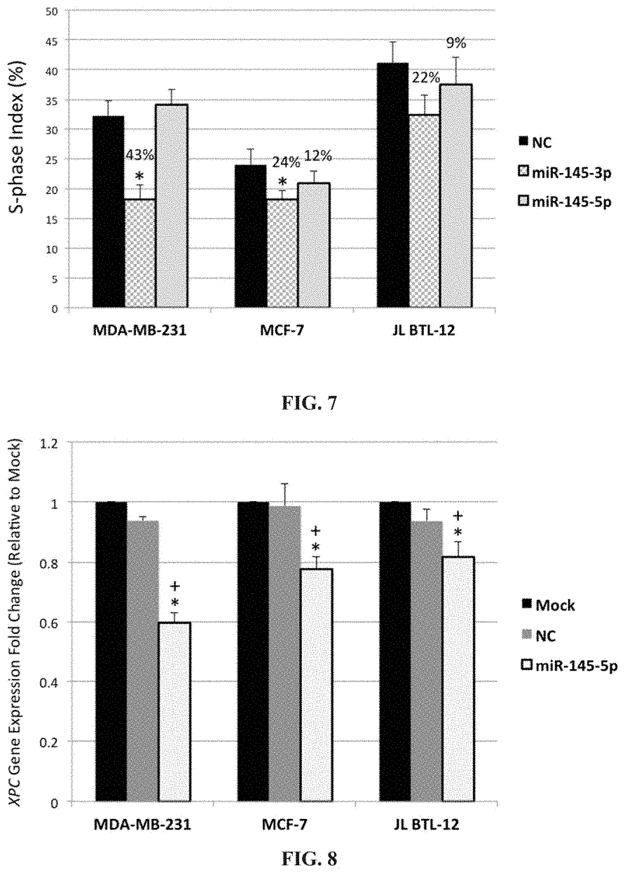

Interestingly, the impact of miR-145-5p on NER capacity was consistently greater than that of miR-145-3p in all three cell lines (FIG. 6). This might be explained by either the number of target NER genes that miR-145-5p has been predicted to target, eight, compared to those targeted by miR-145-3p, five, or simply because the miR-145-5p is the leading strand, that is believed to be responsible for the majority of the functional effect of microRNAs. However, the inventor observed opposite effects of the two strands on cell proliferation: MiR-145-3p reduced the number of S phase nuclei, but miR-145-5p did not in all three cell lines (FIG. 7). This might be because miR-145-3p targets the RPA3 gene, as was validated experimentally in this study. RPA3 was shown to influence the S phase index of one of these three cell lines, MDA-MB-231. The impact of miR-145-5p on replication might suggest that the putative target replication genes for this strand, RPA1 and RPA2, are not true targets. However, this speculation needs to be confirmed experimentally by assessing RPA1 and RPA2 gene and protein expression upon miR-145-5p transfection.

These findings demonstrate the novel role that miR-145 strands play in NER regulation and cell proliferation, making miR-145 a potential therapeutic molecule that might be used to improve genotoxic agent efficacy and ultimately clinical patient outcomes. One additional factor that makes miR-145 a plausible therapeutic molecule is that this microRNA is abundantly expressed in non-diseased breast tissues, and perhaps in all non-diseased human tissues (heart and liver). If this is the case, the intravenous presentation of miR-145 may be a treatment that is not harmful to non-diseased tissues. In fact, miR-145 has been shown to sensitize breast cancer cell lines to chemotherapy agents, including the widely used agent in breast cancer treatment doxorubicin.

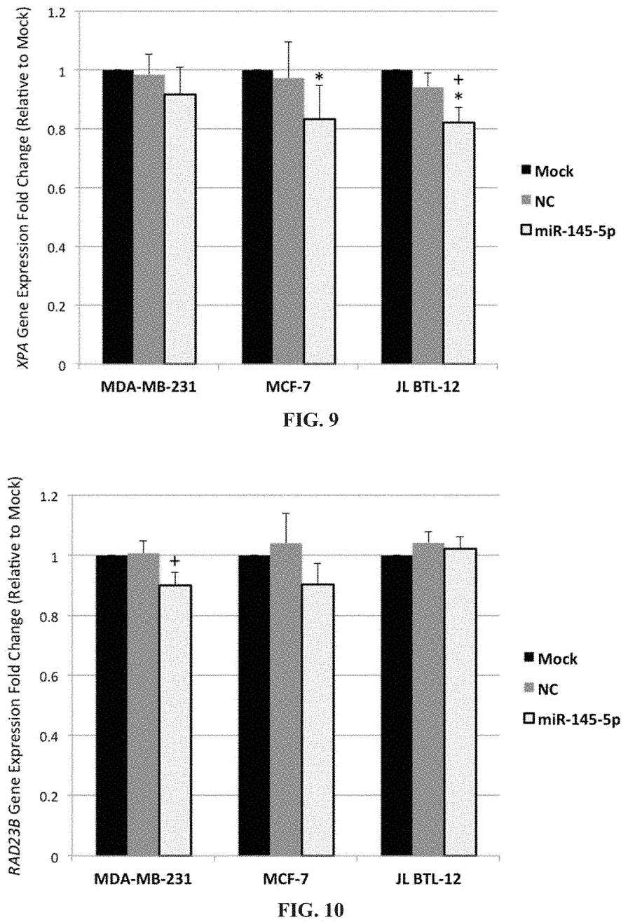

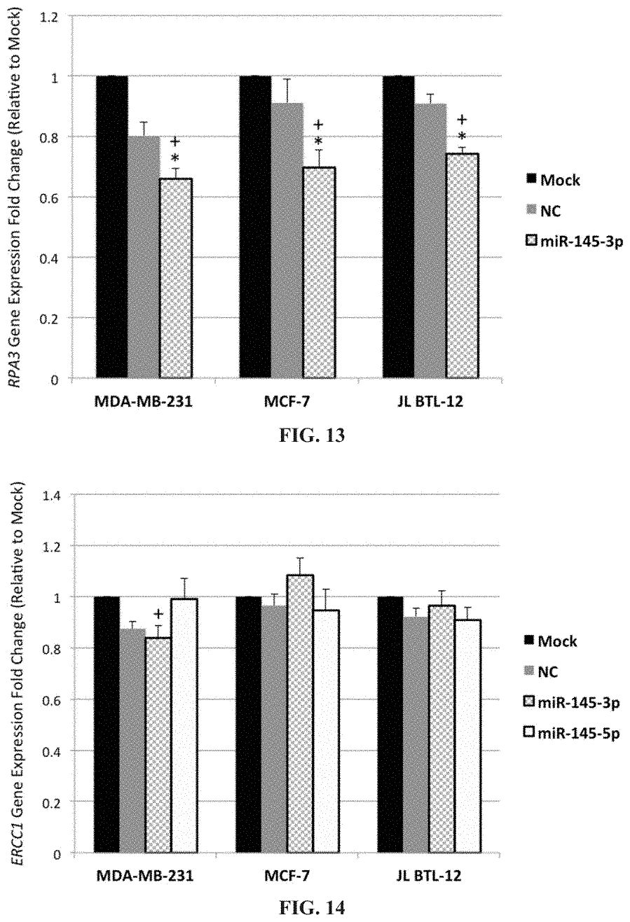

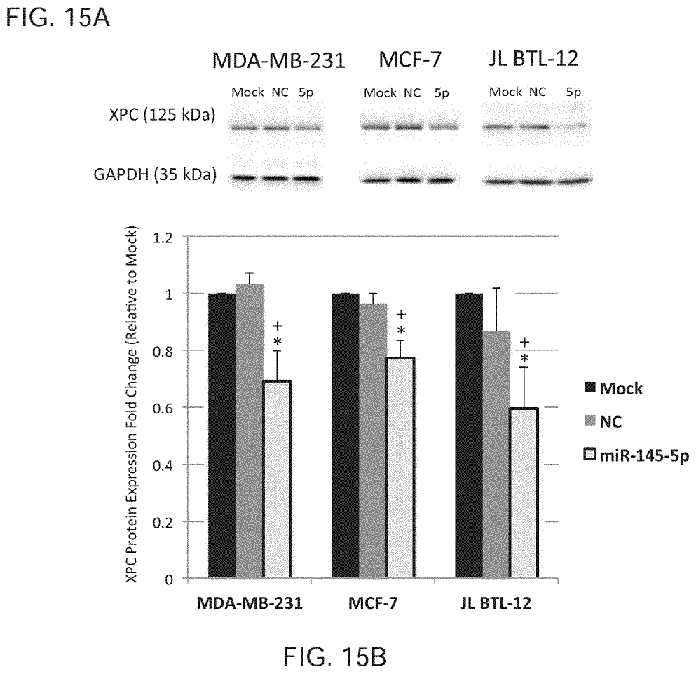

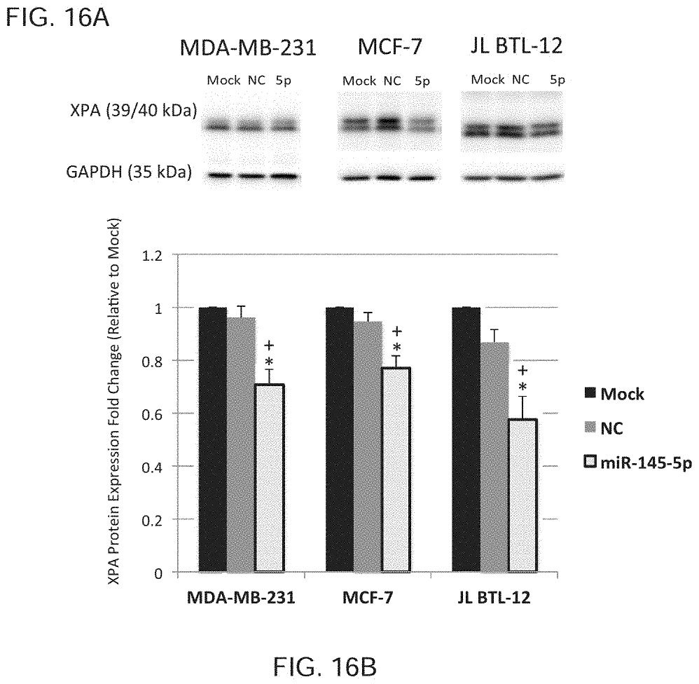

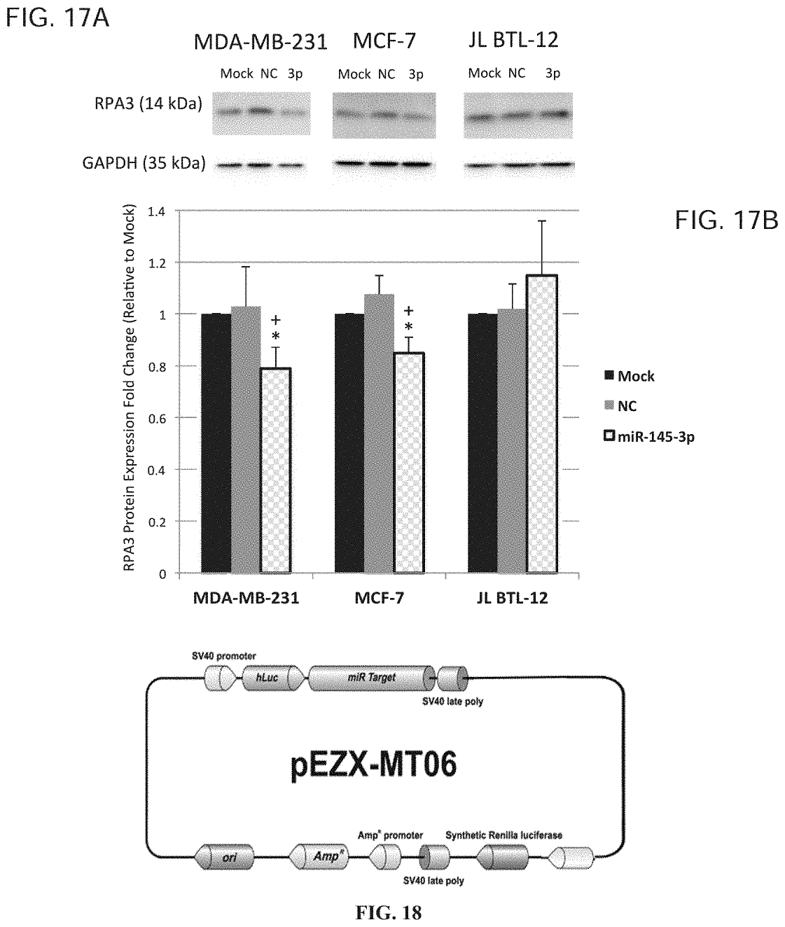

The regulatory role of miR-145-5p and miR-145-3p on five and one predicted putative target NER genes, respectively, were evaluated. Two miR-145-5p target genes XPC (FIG. 8; FIGS. 15A-B) and XPA (FIG. 9; FIGS. 16A-B) and the miR-145-3p target RPA3 (FIG. 13; FIGS. 17A-B) were significantly reduced in expression by miR-145, validate miR145 as a regulator of the NER pathway, and suggesting that this effect on at least these three genes might explain the molecular mechanism by which both strands impacted repair function.

XPC is one of the main recognition proteins in global-genomic NER. XPC recognizes helix-distorting DNA damages and recruits other NER factors to unwind and initiate repair at the DNA damage site. XPC has been found to be mutated in xeroderma pigmentosum (XP), an autosomal recessive hereditary disease in humans characterized by UV-sunlight hypersensitivity and severe predisposition to cancer. Such mutations attenuated the NER function up 75% in XP skin fibroblast compared to healthy skin fibroblasts, illustrating how vital XPC is to NER function. XPC has been found to be overexpressed in several cancer types and to be associated with cisplatin resistance. Cisplatin induces DNA intrastrand crosslinks, which are specifically remediated by NER. These reports suggest that XPC might play an active role promoting NER function in highly chemo-resistant tumors.

XPA is one the first genes shown to be involved in NER. However, the exact mechanistic role that XPA plays in NER is still elusive. It has been speculated to be involved in damage verification, the repair bubble stability, and initiation of incision and excision of 20 to 30-nucleotide segment surrounding the damage. XPA has been found mutated in NER-deficient XP patients. In fact, the loss of NER function caused by XPA mutations is more dramatic compared to any other NER-related mutants in XP, up to 98% loss of repair capacity. This demonstrates that XPA is extremely critical for NER function. Overexpression of XPA has been linked both with worse patient prognosis and chemotherapy resistance. XPA was found to be significantly overexpressed in late-stage breast cancer cell lines compared to non-diseased breast epithelial tissue and stage I breast tumor explants. All these findings demonstrate the vital role that XPA plays in NER, especially in advanced breast tumors characterized by an increase in NER function.

RPA3 is a subunit of the RPA complex that binds to the opposite (undamaged) strand during the repair process. The RPA complex prevents DNA helix re-annealing and protects the undamaged strand from being subjected to degradation by nucleases. RPA3 was the most significantly overexpressed NER gene in the late-stage breast cancer cell lines compared to non-diseased breast epithelial tissue and stage I breast tumor explants. RPA3 upregulation has been found to be involved in radio- and chemotherapy resistance. Silencing RPA3 gene expression significantly reduced NER function and cell proliferation of MDA-MB-231. The dual impact of RPA3 on DNA repair and cell growth makes RPA3 a convincing potential therapeutic target in breast cancer treatment.

The research described herein shows for the first time that microRNAs are important elements regulating NER function, and that their dysregulation in late-stage breast tumors may be an underlying molecular factor in promoting progression-related phenotypes, including gain of NER function. MiR-145 (miRRA) emerged as a candidate microRNA that had potential to regulate NER function. Both the leading strand miR-145-5p and the passenger strand miR-145-3p significantly suppressed NER function in three highly NER-proficient breast cancer cell lines. XPC and XPA were validated experimentally as targets for the leading strand miR-145-5p in the three cell lines while RPA3 was confirmed as a target for the passenger strand miR-145-3p in MDA-MB-231 and MCF-7. This research provides new insights into NER regulation in breast cancer and may potentially establish novel treatment strategies to effectively treat aggressive breast tumors that recur. The putative plan would be to reduce NER capacity in these tumor cells with intravenous miR-145 and use genotoxic chemotherapy with much greater efficacy in the stage III or stage IV scenario.

### Breast Cancer Cell Lines and Culture Explant Selection for MicroRNA Profiling and Culture Conditions

The first aim of this research was to find candidate microRNAs that are dysregulated in breast cancer and might have a role in NER gene regulation. Ten culture explants and cell lines (created in the instant inventor's laboratory) as well as commercially available cell lines representing three groups: non-diseased breast epithelial tissues, stage I breast tumors, and late-stage breast tumors were selected. These three groups were included to be able to identify microRNAs that might be involved in both of the two NER phenotypes identified in breast cancer; a loss-of-function in early-stage breast cancer followed by a gain-of-function in later stages. The non-diseased breast epithelial tissue group was comprised of three representative normal breast tissue culture cell lines: JL BRL-6 p13, JL BRL-14 p19, and JL BRL-36 p24. The stage I breast tumor group included three representative stage I breast tumor culture explants and cell lines: JL BTL-4 p14, JL BTL-8 p12, and JL BTL-33 p14. The late-stage breast tumor group consisted of a stage III breast tumor cell line, JL BTL-12 p16, and three established breast cancer cell lines: MCF-7 p18, MDA-MB-231 p19, and BT-20 p15.

Non-diseased breast tissue cell lines, breast tumor explants and cell lines, established breast cancer cell lines were maintained in culture.

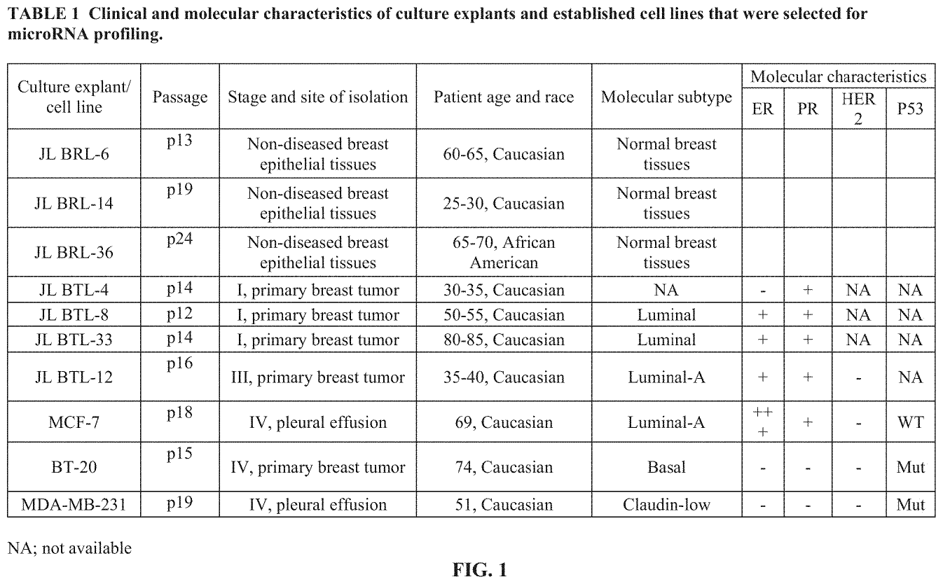

Table 1, as shown in FIG. 1, charts the clinical and molecular characteristics of culture explants and established cell lines that were selected for microRNA profiling.

Isolation of Total RNA (Including microRNA)

Total RNA, including microRNA, was harvested from the 10 selected culture explants and established cell lines using the miRNeasy mini kit (Qiagen) (Cat #217004) following the manufacturer's protocol.

Total RNA concentration and purity for all samples was determined. The harvested RNA samples were run in mini-formaldehyde northern RNA gels to evaluate RNA quality and to allow for adjustment of the total RNA concentration that was initially measured using the spectrophotometer.

### MicroRNA Expression Analysis (Nanostring)

A hundred ng of total RNA, including the microRNAs, from each sample were used to perform Nanostring microRNA expression that was carried out at the NanoString headquarters (Seattle, Wash.) using the nCounter Human v3 microRNA expression panel. This panel contained 800 probes representing 800 well-known human microRNAs. microRNA expression data was received as .RCC files and processed using nSlover® software 2.0v (Nanostring, Inc.).

MicroRNA expression files underwent three layers of data processing. The first layer excluded the background noise by utilizing the highest signal count of six negative control probes. The highest negative control count was subtracted from all 800 microRNA probe counts for each sample. The second layer consisted of normalizing the background-subtracted probe count values using six positive control probes that account for the technical variation of performing microRNA expression assays. The last layer of data processing was to normalize all the 800 microRNA probe counts to the geometric mean of five housekeeping gene expressions: RPLP0, RPL19, GAPDH, B2M, and B-Actin, as presented as controls on the microRNA chip.

MicroRNA expression values of the non-diseased breast epithelial tissue, stage I breast tumor, and late-stage breast tumor groups were examined statistically using a one-tailed, unpaired student's t-test. P values<0.05 was considered statistically significant. Since microRNA is an inhibitory mechanism, the ideal microRNA candidate expression would be inversely correlated with NER function in these groups; significantly overexpressed in the stage I tumors compared to non-diseased breast tissues and significantly under-expressed in late-stage breast tumors compared to stage I breast tumors.

Unsupervised hierarchical clustering analysis utilizing the 800 microRNA expression signature of the ten culture explants and cell lines was performed using the nSolver software (Nanostring, Inc). The Euclidean algorithm and the average linkage method were used to generate dendrograms.

### MicroRNA Putative Binding Sites in NER Genes Using MicroRNA Predicted Target Databases

Concurrently with bench work profiling microRNA expression in a selected series of non-diseased breast and breast tumor explants/established cell lines, the most commonly downregulated microRNAs in breast cancer, that had been reported in at least three independent studies, were identified. Putative binding sites for these microRNAs in the 20 NER canonical genes were searched using three publically-available microRNA predicted target databases: miRanda, TargetScan, and miRWalk. Each database utilizes a different computational algorithm to identify and rank putative microRNA/mRNA interactions.

Choosing the candidate microRNA for further experimental validation was based on three main criteria: the microRNA was significantly under-expressed in late-stage breast cancer compared to early breast cancer in the Nanostring analysis, the microRNA was predicted to bind to as many as NER genes as possible with good prediction scores in miRanda and TargetScan or significant P values in miRWalk, and target NER genes were prioritized based on their expression in late-stage breast cancer (overexpressed) and how crucial and specific the genes were to the NER pathway.

MicroRNA-145-3p Expression (microRNA RT PCR)

After miR-145 was selected for further experimental investigation, miR-145-3p (the passenger strand) was examined in the ten selected culture explants and cell lines using microRNA RT-PCR, since it was not included in the Nanostring nCounter Human v3 microRNA expression panel. Unlike the reverse transcriptase step in the traditional RT-PCR, only mature transcripts of a single microRNA are converted to copy DNA (cDNA) transcripts using reverse transcription primers that are specifically designed to that microRNA.

Ten ng of total RNA of each sample was used to generate copy DNA transcripts of miR-145-3p as follows: 10 ng of total RNA was diluted in five μl of DEPC H₂O then mixed with 3 μl of 5×RT miR-145-3p primers and 7 μl of RT-PCR master mix that was prepared beforehand by mixing 0.15 μl of 100 mM dNTPs, one μl of MultiScribe® reverse transcriptase, 1.5 μl of 10×RT buffer, and 4.35 μl of DEPC H₂O. MiR-145-3p cDNA transcripts were generated in three thermal steps: 16° C. for 30 minutes, 42° C. for 30 minutes, then 85° C. for five minutes using the mastercycler.

The predesigned miR-145-3p Taqman® gene expression assay (Invitrogen®) (Cat #4427975; ID #002149) was used to quantify miR-145-3p expression in the samples. In addition, the predesigned RNU24 Taqman® gene expression assay (Invitrogen®) (Cat #4427975; ID #001001) was used to quantify the abundantly expressed non-coding small nucleolar RNA RNU24 that was used to normalize miR-145-3p expression. Three technical replicates were run for each sample. The RT-PCR reaction volume of 20 ul was prepared for each technical replicate by mixing 1 μl of 20× Taqman gene expression assay, 10 μl of 2× Taqman gene expression master mix, 1.33 μl of cDNA (0.89 ng), and 7.67 μl of DEPC H₂O. Ten percent excess volume was considered to compensate for volume loss from pipetting. The 20 ul RT-PCR reaction volume of each replicate was transferred to a RT-PCR 96-well plate then sealed with an adhesive cover to prevent cross contamination among wells. The well plate was loaded into the StepOnePlus Real times qPCR system. The qPCR amplification process was run using three thermal steps 50° C. for 2 minutes, 95° C. for 10 minutes, then 40 cycles of 95° C. for 15 seconds followed by 60° C. for one minute.

The average of RNU24 cycle threshold (C_{T}) value was subtracted from the average of miR-145-3p C_{T} value of the technical replicates to obtain a ΔC_{T} value for each sample. ΔC_{T} values of the samples in the reference group (non-diseased breast tissue) were averaged then subtracted from ΔC_{T} values of the 10 selected culture explants and cell lines to obtain logarithmic relative gene expression values (ΔΔC_{T}). ΔΔC_{T} values were exponentially transformed using the equation 2^{-ΔΔC}^{_{T}} to obtain relative fold change expression values. MiR-145-3p expression in the three groups was evaluated statistically using a one tailed, unpaired student's t test. A P value<0.05 was considered statistically significant.

Cell Line Selection and Experimental Design (miR-145 Experiments)

MDA-MB-231, MCF-7, and IL BTL-12 were selected to investigate the effect of miR-145 on NER function and target NER genes.

Both miR-145 strands, miR-145-5p and miR-145-3p, were independently introduced into the three cell lines using the lipofectamine transfection technique. Five transfection treatment groups were assigned for each cell line: blank, mock-treated, negative (scrambled sequence) control, miR-145-3p, and miR-145-5p. The blank sample was a non-treated sample that was used to ensure that the optimal culture conditions were maintained throughout the experiment. The mock sample was transfected with only the transfection vehicle, Lipofectamine®. The negative control sample was transfected with a transfection complex composed of lipofectamine and a (FITC)-tagged scrambled RNA that mimic the structure of the mature microRNAs. The miR-145-3p sample was transfected with the passenger strand miR-145-3p transfection complex consisting of mature, synthetic miR-145-3p duplex (Invitrogen®) (Cat #4464066; ID #MC13036) and lipofectamine. The miR-145-5p sample was transfected with the leading strand miR-145-3p transfection complex consisting of mature, synthetic miR-145-5p duplex (Invitrogen®) (Cat #4464066; ID #MC11480) and lipofectamine.

### Lipofectamine Transfection

Two miR-145-3p and miR-145-5p doses, 60 and 120 pmole, were used to optimize transfection conditions for each cell line. The 120 pmole dose was found to be toxic to the three cell lines, as manifested by cell death. Therefore, the research proceeded with the 60 pmole dose.

### Unscheduled DNA Synthesis Assay

The impact of both miR-145 strands, miR-145-5p (leading strand) and miR-145-3p (passenger strand), on NER function was assessed in all three cell lines. Forty thousand cells of MDA-MB-231 and MCF-7 were each plated in each chamber of a two-chamber Nunc chamber slides 36 hours prior to transfection. Three treatment groups were included in the UDS experiments negative control (scrambled RNA), miR-145-3p transfected, and miR-145-5p transfected. Three replicate slides were used for each treatment group in each cell line. Transfection efficiency was examined 24 hours after transfection and the UDS assay was performed 48 hours after transfection.

One tailed, paired or unpaired student's t test was used in order to identify the miR-145-3p and miR-145-5p transfected samples that had a significant decrease in NER function compared to the negative control samples. P values<0.05 was considered statistically. Final assessment of the impact of these microRNAs was done by expressing these data as a proportion of foreskin fibroblast (standard in the field and allows for comparison with future experiments when these miR-145 experiments are repeated).

S-phase indices of the cells in the three treatment groups were calculated to assess the impact of both miR-145 strands transfection on replication (proliferation). S-phase indices were evaluated by calculating the S-phase cell percentage on all counted microscopic fields on the irradiated sides. One tailed, paired student's t test was used in order to identify the miR-145-3p and miR-145-5p transfected samples that had a significant decrease in S-phase indices compared to the negative control samples. A P value<0.05 was considered statistically significant.

### Total RNA Isolation and Northern RNA Gels

Total RNA was harvested from four treatment groups: mock, negative control, miR-145-3p, and miR-145-5p using the RNeasy mini kit (Qiagen) 48 hours after transfection. The extraction process and RNA concentration and purity determination were performed. The harvested RNA samples were run in northern RNA gels to evaluate RNA quality and adjust the total RNA concentration that was initially measured using the spectrophotometer.

### RT-PCR

Five miR-145-5p target NER genes (XPA, XPC, RAD23B, ERCC6, and GTF2H4), and one miR-145-3p target NER gene (RPA3) were selected for experimental validation to evaluate the potential regulation of these genes by miR-145 strands. Selection of these genes was based on several factors. These factors were prioritized as the following: the significance of the predicted interactions obtained from microRNA predicted target databases, their expression in late-stage breast cancer, the importance of the genes to the pathway as determined by the impact of the NER gene on the pathway when it is mutated, and then the locations of the miR-145 seed matches. An off-target NER gene, ERCC1, was also included to evaluate any indirect effects of either miR-145 strands that might affect the NER pathway. The effect of miR-145 transfection on the expression of these seven genes was evaluated in five independent experiments for each cell line using RT-PCR.

Predesigned, commercially available Taqman® gene expression assays (Invitrogen®) of the following were used to quantify the miR-145 target genes; RPA3 (Cat #448892; ID #Hs01047933_g1), XPA (Cat #4331182; ID #Hs00902270m1), XPC (Cat #4331182; ID #Hs00190295_m1), ERCC6 (Cat #4453320; ID #Hs00972920_m1), RAD23B (Cat #448892; ID #Hs01011338_g1), and ERCC1 (Cat #4453320; ID #Hs01012158_m1). The custom made Taqman® gene expression assay (Cat #4441114; ID #AJHSOKZ) was used to quantify GTF2H4 since there was no predesigned prime for this gene. The prime was made based on Invitrogen® standard specifications for designing Taqman® gene expression assays. A Predesigned Taqman® gene expression assay (Invitrogen®) (Cat #4453320) (ID #Hs02758991_g1) was used to quantify the housekeeping gene GAPDH that was used to normalize all the six gene expression data. Reverse transcription, PCR amplification, and data analyses were performed.

One tailed, paired student's t test was used in order to identify miR-145-3p and miR-145-5p transfected samples that had a significant decrease in the selected gene expression compared to both mock and negative control samples. A P value<0.05 was considered statistically significant.

### Total Protein Isolation and Quantification

The impact of miR-145 transfection on protein expressions of two proteins, XPC and RPA3 was evaluated. These proteins were chosen because the expression of the corresponding genes was shown to be significantly reduced upon miR-145 transfection in all three cell lines. The effect of miR-145 on XPA protein expression was also examined in the three cell lines. XPA was a top potential candidate target since it was the only putative target gene that was confirmed using the three microRNA predicted databases. Although XPA gene expression was not significantly reduced in MDA-MB-231 and MCF-7, the impact of miR-145-5p might be seen only on XPA protein expression, not at the gene transcript level.

Four independent experiments were performed in order to evaluate the effect of miR-145 on the target protein expression in each cell line. Total protein was extracted from samples 48 hours after transfection. Sample dishes were washed with ice cold PBS then 100 μl of radioimmunoprecipitation assay (RPIA) lysing buffer (Life Technologies®) (Cat #89900) mixed with Halt® protease inhibitor cocktail (Life Technologies®) (Cat #78438) was added to each dish. Cell lysate was removed after scraping cells from the dishes and transferred to a 1.5 ml tube then maintained under a constant shaking for 30 minutes at 4° C. Sample tubes were centrifuged at 12,000 RPM at 4° C. for 20 minutes. The cell lysate solution containing total protein was transferred to another tube while the pellet was discarded. Protein samples were flash frozen and stored in the −80° C. freezer.

Total protein was quantified using the colorimetric Pierce® bicinchoninic acid (BCA) assay kit (Life Technologies®) (Cat #89900). Protein samples were diluted 10 fold in DEPC H₂O. Serial dilutions of bovine serum albumin (BSA) standards were prepared to range from 25 to 2000 μg/ml. Twenty-five μl of three replicates of each diluted unknown protein sample and BCA standard were added to a 96-well plate then mixed with 200 μl of the detection reagent, which was prepared beforehand by mixing BCA reagent A with BCA reagent B in a ratio of 1:8. Two hundred μl of three replicates of the detection reagent was added to the well plate and severed as blank samples used to assess the background noise. The 96-well plate was incubated at 37° C. for 30 minutes and then the absorbance was measured at a wavelength of 562 nm using Synergy H1 plate reader (BioTek®).

The average of the background absorbance values of the blank replicates was subtracted from the 562 nm reading of all individual BSA standard and unknown sample replicates. A standard curve was obtained by plotting the average background-corrected absorbance value for each BSA standard against its concentration. The standard curve was used to determine the total protein concentration of each unknown sample.

### Western Blotting

Protein samples were run on 8% sodium dodecyl sulfate-polyacrylamide gels (SDS-PAGE) to detect and quantify XPA (38 kDa) and XPC (125 kDa) proteins while 12.5% SDS-PAGE was used to quantify the RPA3 protein since it is a low molecular weight protein (14 kDa). Five control samples were run on each acrylamide gel; five and 20 μg of total protein from foreskin fibroblasts, which served as a standard, used to normalize sample protein band intensities across different gels; and three serial total protein amounts five, 10, 20 μg of untreated sample for each cell line, which were used to examine the sensitivity of antibody binding. Standardization to the foreskin fibroblasts band intensities can be used to enable comparisons with past and future experiments.

Twenty μg total protein of each sample was diluted in seven μl of RIPA buffer, then mixed with 2.33 μl 4× Laemmli loading dye (BioRad®) (Cat #1610747). The mixed protein samples were denatured at 70° C. for 10 minutes in the mastercycler, then loaded onto the gel. five μl of Precision® plus protein dual color molecular weight standards (BioRad®) (Cat #1610374) was mixed with 4.33 μl RIPA buffer then loaded onto the gel; this was done to ensure that the protein bands were the right sizes for their respective target proteins. Electrophoresis of 8% and 12.5% acrylamide gel was carried out at 150 volts in a running buffer (25 mM Tris, 192 mM glycine, 3.5 mM SDS, pH=8.3) at 4° C. for one hour and 1.5 hours, respectively.

After gel electrophoresis, proteins were transferred to nitrocellulose membranes with pore size of 0.2 μm (BioTrace®) (Cat #66489) in the presence of a transfer buffer (25 mM Tris, 192 mM glycine, 20% methanol, pH=8.3). For XPA and XPC analysis, the transfer was carried out at 20 volts for 16 hours at 4° C., while for RPA3 transfer was performed at 60 volts for an hour at 4° C. These conditions were optimized to obtain a successful transfer for these proteins. The membrane was washed with Tris-buffered saline (TBS) for 10 minutes then blocked with 5% blotting grade non-fat milk (BioRad®) (Cat #1706404XTU) dissolved in Tris-buffered saline, 0.1% Tween 20 buffer (TBST) under constant shaking for an hour at room temperature. After blocking, the membrane was washed twice with TBST under vigorous shaking for 10 minutes each, then incubated with XPA, XPC, RPA3, and GAPDH primary antibodies for 16 hours at 4° C. under constant shaking. Antibody specifications and conditions are summarized in Table 2.

Protein band intensities were measured using Image Studio Lite software 5.2.5v (LI-COR®). Membrane background signal was assessed above and below each band then subtracted from the total integrated intensity of that band. GAPDH band intensity was used to normalize XPA, XPC, and RPA3 band intensity values. GAPDH-corrected band intensity values of negative control, miR-145-3p, and miR-145-5p transfected samples were expressed relative to the mock sample in each experiment. The target protein intensity evaluated in four independent experiments. One tailed, paired student's t test was used in order to identify miR-145-3p and miR-145-5p transfected samples that had a significant decrease in the selected protein expression compared to both mock and negative control samples. A P value<0.05 was considered statistically significant.

TABLE 2
Antibody specifications and conditions used in Western analyses

| | | | | Protein |
|---|---|---|---|---|
| | | | | detected size |
| Protein | Source | Type | Dilution | on SDS-PAGE |
| XPA | Sigma-Aldrich ® | Rabbit | 1:2000 | Two bands |
| | (Cat# X1254) | polyclonal | in TBST | (39 and 40 kDa) |
| XPC | Cell Signaling ® | Rabbit | 1:1000 | 125 kDa |
| | (Cat# 12701) | polyclonal | in TBST | |
| RPA3 | Abcam ® | Rabbit | 1:1000 | 14 kDa |
| | (Cat# ab97436) | polyclonal | in TBST | |
| GAPDH | Cell Signaling ® | Rabbit | 1:2000 | 35 kDa |
| | (Cat# 5174) | monoclonal | in TBST | |

### MicroRNA Signature in Breast Cancer

The overall microRNA expression pattern was evaluated in the three groups: non-diseased breast epithelial tissue (JL BRL-6, JL BRL-14, JL BRL-36), stage I breast tumors (JL BTL-4, JL BTL-8, JL BTL-33), and late-stage breast tumor (JL BTL-12, MCF-7, MDA-MB-231, BT-20) using unsupervised hierarchical clustering.

FIG. 2 is a dendrogram showing the resulting microRNA expression patterns of non-diseased breast tissue, stage 1 breast tumor tissue, and late-stage breast tumor tissue groups. The unsupervised hierarchical clustering analysis was performed utilizing 800 probes representing 800 microRNAs that were measured using Nanostring nCounter Human v3 microRNA expression panel. The dendrogram was generated using the Euclidean algorithm and average linkage methods. The analysis revealed two main clusters; the first cluster contained a mix of the non-diseased breast cell lines (JL BRL-6, JL BRL-14, and JI-BRL-36) and the stage I tumor explants and cell lines (JL BTL-4, JL BTL-8, JL BTL-33) while the other cluster included the late-stage breast tumor derived cell lines (MDA-MB-231, MCF-7, and BT-20) and the late-stage explant JL BTL-12. The heat map is also shown in the dendrogram (FIG. 2).

These results indicate that late-stage cancer had a distinctive microRNA expression signature that was quite different from the non-diseased and stage I tumors. In addition, the dysregulation of microRNAs in late-stage breast cancer might be an underlying molecular factor associated with breast cancer progression.

A clear separation between non-diseased breast tissue and stage I tumor explants in the first cluster, based on their overall expression of 800 microRNAs, was not observed. However, it was hypothesized that a supervised analysis of a specific subset of microRNAs might be able to differentiate them from each other.

### Putative Candidate MicroRNAs Involved in NER Regulation

Since microRNAs are inhibitory small molecules, the ideal microRNA candidate that is involved in NER regulation, if there is only one of them, should show expression that is inversely correlated with NER function in these groups. In other words, it should be significantly overexpressed in stage I tumors compared to non-diseased breast, and significantly under-expressed in late-stage breast tumors compared to stage I breast tumors (FIG. 3, selection of the candidate microRNA). The red line in FIG. 3 represents the hypothetical expression of the ideal candidate microRNA.

First, the expression of 800 microRNAs in the stage I tumor explants and cell lines JL BTL-4, JL BTL-8, and JL BTL-33 was compared to that of the non-diseased breast reduction epithelial cell lines JL BRL-6, JL BRL-14, JL BRL-36. Only one microRNA, miR-100-5p, was found that was significantly upregulated 1.69 fold in the stage I tumor group compared to non-diseased breast tissue group (P=0.044) (Table 3). The microRNA expression profile of JL BRL-6 and JL BTL-33 were noticeably different when compared with the cell lines within their groups, non-diseased tissue and stage I tumors, respectively. These variations in microRNA expression within each group might explain why only one overexpressed microRNA miR-100-5p reached statistical significance. A better sample selection of other non-diseased and stage I explants and cell lines might help to identify a larger set of candidate microRNAs. Another possibility is that microRNA might not be the major epigenetic regulatory mechanism involved in the loss of NER function in stage I breast tumors as in the gain of NER function in late-stage breast tumors. A third possibility is that one microRNA cannot explain both the loss of NER in tumor formation and the gain of NER in tumor progression.

When the microRNA expression pattern of the stage I tumor group was compared to the late-stage group, 45 microRNAs were found to be significantly under-expressed (P<0.05), ranging from 5093-fold (miR-145-5p) to 2.34-fold (miR-29a-3p). These data suggested that a microRNA-based mechanism might be heavily involved in breast cancer progression-related phenotype.

One microRNA was the only microRNA that met the ideal candidate microRNA expression pattern, significantly overexpressed in early stage and significantly under-expressed in late stage. However, this microRNA was predicted to target only one NER gene, CDK7, using the three microRNA target databases, which is a very low number for computational target genes and it lacks specificity for NER. The inventors were more interested in microRNAs with the ability to regulate more than just one NER gene at the same time. Therefore, the focus of the study/research shifted to finding microRNAs whose expressions were significantly lost or decreased in late-stage tumors and might explain the increase in NER function in such tumors.

TABLE 3
Upregulated microRNA in stage I tumor explants
and cell lines compared to non-diseased breast cell lines.

| | | | Fold change | |
|---|---|---|---|---|
| | Non-diseased | Stage I breast | (relative non- | |
| microRNA | breast (n = 3) | tumor (n = 3) | diseased) | P value |
| miR-100-5p | 7967.36 | 13495.64 | 1.69 | .044 |

Predicted Putative Binding Sites of Candidate microRNA in NER Genes

Concurrently with profiling microRNA expression in the selected culture explants and cell lines, the instant inventors searched the microRNA literature for microRNAs under-expressed in breast cancer and examined their potential to target NER genes in three publically available predicted microRNA target databases miRanda, TargetScan, and miRWalk. 11 microRNAs that were reported as downregulated in breast cancer in at least three independent studies were found (Table 4). The number of putative target NER genes for these microRNAs varied from four to 14 genes. Six out of the identified 11 microRNAs had seeding regions for at least 10 NER genes. These data indicated that microRNAs have the potential to be important regulatory elements in NER genes regulations that ultimately impact the NER function.

MiR-145 emerged as a top candidate microRNA that might be involved in NER regulation.

TABLE 4
The microRNAs most commonly under-expressed in breast cancer

| # | microRNA | Reference |
|---|---|---|
| 1 | let-7a | (Iorio et al., 2005b; Kim et al., 2012b; Liu et al., |
| | | 2015; Volinia et al., 2006) |
| 2 | let-7d | (Chang et al., 2011; Iorio et al., 2005; Volinia |
| | | et al., 2012) |
| 3 | let-7f | (Iorio et al., 2005b; Patel et al., 2011; Zhao |
| | | et al., 2011) |
| 4 | miR-204 | (Iorio et al., 2005b; Li et al., 2014; Liu & Li, 2015; |
| | | Wang et al., 2015; Zhu et al., 2014) |
| 5 | miR-143 | (Farazi et al., 2011; Iorio et al., 2005b; Ng et al., |
| | | 2014; Yan et al., 2014) |
| 6 | miR-145 | (Farazi et al., 2011; Iorio et al., 2005b; Spizzo |
| | | et al., 2010a; Volinia et al., 2006; Yan et al., 2014) |
| 7 | miR-101 | (Iorio et al., 2005b; Wang et al., 2014; Wang et al., |
| | | 2012) |
| 8 | miR-34a | (Iorio et al., 2005b; Li et al., 2013; Peurala et al., |
| | | 2011; Yang et al., 2013) |
| 9 | miR-34b | (Iorio et al., 2005b; Lee et al., 2011; Liu et al., |
| | | 2015) |
| 10 | miR-34c | (Iorio et al., 2005b; Liu et al., 2015; Yang et al., |
| | | 2013; Yu et al., 2012; Zhu et al., 2014) |
| 11 | miR-125a | (Guo, Wu, & Hartley, 2009; Iorio et al., 2005b; |
| | | Scott et al., 2007; Zhu et al., 2014) |

### MiR-145 Expression in Late-Stage Breast Cancer

The leading strand of miR-145, miR-145-5p, was ranked the most under-expressed microRNA among 800 microRNAs evaluated in late-stage breast tumor cell lines compared to the stage I tumor group (P=0.005) (FIG. 4). FIG. 4 is a graph showing the leading strand miR-145-5p expression in the late-stage breast tumor tissue group compared to the stage 1 breast tumor tissue group using the Nanostring microRNA expression panel. MiR-145-5p expression was expressed relative to the average expression of the stage I breast tumor explants and cell lines. The miR-145-5p expression fold change of the late-stage group is shown. Error bars represent the standard error of each group. P<0.05 is considered statistically significant using one-tailed unpaired student's t test.

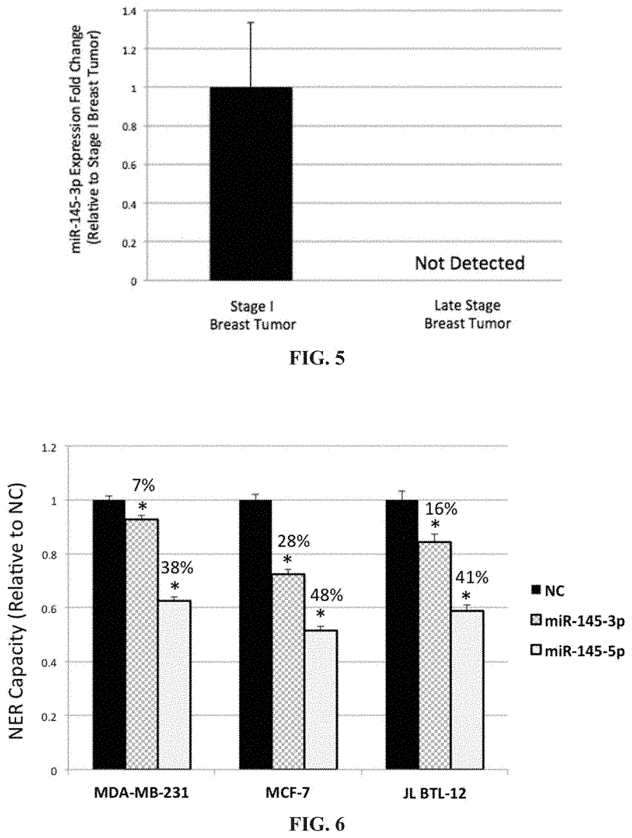

The passenger strand, miR-145-3p, showed a similar expression trend when its expression was assessed using RT-PCR. MiR-145-3p was abundantly expressed in stage I tumor explants and cell lines but it was not detected in the stage III breast tumor cell line JL BTL-12 and the commercial late stage derived cell lines (FIG. 5). FIG. 5 is a graph showing the passenger strand miR-145-3p expression in the late-stage breast tumor tissue group compared to the stage 1 breast tumor tissue group using microRNA RT-PCR. MiR-145-3p expression was expressed relative to the average expression of the stage I breast tumor explants and cell lines. MiR-145-3p was abundantly expressed in the stage I tumor group but it was not detected in the late-stage breast tumor group. Error bars represent the standard error of the stage I tumor group.

### Unscheduled DNA Synthesis Assay

The impact of both miR-145 strands after transfection on NER capacity in the three late-stage breast cancer cell lines was investigated using the functional unscheduled DNA synthesis assay. Three miR-145-3p and miR-145-3p transfected slides were compared to three negative control transfected slides for each cell line. NER function was reduced significantly after both the leading strand miR-145-5p and the passenger strand miR-145-3p were transfected, compared to negative control transfected cells in all the three cell lines (FIG. 6). FIG. 6 is a graph showing the impact of miR-145 on nucleotide excision repair (NER) capacity in MDA-MB-231, MCF-7, and JL BTL-12 breast cancer-derived cell lines. MiR-145-3p transfected cells (MDA-MB-231 [n=506], MCF-7 [n=300], JL-BTL-12 [n=300]) and miR-145-5p transfected cells (MDA-MB-231 [n=420], MCF-7 [n=300], JL-BTL-12 [n=300]) had consistent, significant, reductions in NER function compared to negative control cells (MDA-MB-231 [n=402], MCF-7 [n=300], JL-BTL-12 [n=300]) in all the three late stage breast cancer derived cell lines (P<0.001). NER capacity is expressed relative to negative control in each cell line. Error bars represent standard error of the pooled grain counts over the counted nuclei in three slides in each transfection group. P<0.05 is considered statistically significant using one-tailed paired or unpaired student's t test. NC; negative control group.

In MDA-MB-231, NER function was significantly reduced by 7.22% (n=506, P<0.001) and 37.51% (n=420, P<0.001) in miR-145-3p and miR-145-5p transfected cells, respectively, when compared to negative control transfected cells (n=402). MCF-7 miR-145-3p and miR-145-5p transfected cells showed significantly decreased NER capacity by 27.63% (n=300, P<0.001) and 48.41% (n=300, P<0.001) compared to negative control treated cells (n=300), respectively. Lastly, JL BTL-12 miR-145-3p and miR-145-5p transfected cells had a significant reduction in NER function by 15.57% (n=300, P<0.001) and 41.25% (n=300, P<0.001) in relative to negative control transfected samples (n=300).

These results suggest that both miR-145 strands may act to regulate NER function in late breast cancer cell lines. Interestingly, the impact of miR-145-5p on NER capacity was consistently greater than that of miR-145-3p in all three cell lines. This might be explained by either the number of target NER genes that miR-145-5p was predicted to target, eight, compared to miR-145-3p, five, or simply because the miR-145-5p is considered the leading strand, and that is believed to be responsible for the majority of the functional effects of miRNAs.

### S-Phase Index Assay

Both miR-145 strands putatively target replication genes. The leading strand, miR-145-5p, targets RPA1 and RPA2 whereas the passenger strand targets RPA3. Therefore, the impact of miR-145-3p and miR-145-5p transfection on cell proliferation was examined by determining S phase indices in the three breast cancer cell lines. The UV unirradiated side of the slide was used for a clean count of nuclei incorporating radiolabel through DNA synthesis without the background of DNA damage repairing nuclei. MiR-145-3p transfected slides had a decrease in S phase index by 43%, 24%, and 22% in MDA-MB-231, MCF-7, and JL BTL-12, respectively (FIG. 7). This reduction reached statistical significance in MDA-MB-231 (P=0.005) and MCF-7 (P<0.001), but not JL BTL-12 (P=0.072). MiR-145-5p did not have a significant impact on S phase indices in the three cell lines. FIG. 7 is a graph showing this impact of miR-145 on cell proliferation in MDA-MB-231, MCF-7, and JL BTL-12 breast cancer-derived cell lines. S-phase index was calculated for each treatment group in three breast cancer derived cell lines. As noted, S-phase indices were significantly reduced in miR-145-3p transfected slides relative to negative control slides in MDA-MB-231 (P=0.005) and MCF-7 (P<0.001). The percentages of S-phase index reduction in miR-145-3p and miR-145-5p transfected slides compared to negative control are shown. Error bars represent standard errors of the pooled s-phase indices of three slides in each treatment group. P<0.05 was considered statistically significant using one-tailed paired student's t test. These results suggest that miR-145 might suppress breast cancer cell proliferation, and that the passenger strand miR-145-3p is responsible for such suppression.

### MiR-145 Target Gene Expression

After it was demonstrated that both miR-145 strands modified NER function in late stage breast cancer, the effect of both strands on gene expression of a selected group of predicted target NER genes was examined to confirm the molecular mechanisms by which miR-145 regulates NER function.

XPC gene expression was significantly reduced by miR-145-5p transfection in all three cell lines (FIG. 8). FIG. 8 is a graph showing XPC gene expression regulation by miR-145-5p in MDA-MB-231, MCF-7, and JL BTL-12 breast cancer-derived cell lines. XPC gene expression is significantly reduced in miR-145-5p transfected samples compared to mock and negative control samples in the three cell lines. The fold change in XPC gene expression is expressed relative to the mock samples. Error bars represent standard error for five independent experiments for each cell line. + and * indicate significant decreases in XPC gene expression vs. mock and negative control samples, respectively. P<0.05 was considered statistically significant using one-tailed paired student's t test. NC; negative control group. In MDA-MB-231, it was reduced by 40.33% (P<0.001) and 36.39% (P<0.001) by miR-145-5p compared to mock and negative control samples, respectively. In MCF-7, XPC was decreased by 22.41% (P=0.003) and 21.37% (P=0.004) by miR-145-5p compared to mock and negative control samples, respectively. In JL BTL-12, miR-145-5p transfected samples had 18.24% (P=0.011) and 12.69% (P=0.009) decrease in XPC gene expression relative to mock and negative control samples, respectively. These results suggest that miR-145-5p might play an important role in XPC gene regulation by triggering mRNA degradation.

XPA gene expression was significantly reduced in JL BTL-12 after transfection with miR-145-5p by 17.97% (P=0.014) and 12.96% (P=0.015) compared to mock and negative control samples, respectively (FIG. 9). FIG. 9 is a graph showing XPA gene expression regulation by miR-145-5p in MDA-MB-231, MCF-7, and JL BTL-12 breast cancer-derived cell lines. As noted, XPC gene expression is significantly reduced in miR-145-5p transfected samples compared to mock and negative control samples only in the JL BTL-12 cell line. The fold change in XPA gene expression is expressed relative to the mock samples. Error bars represent standard errors over five independent experiments for each cell line. + and * indicate significant decreases in XPA gene expression vs. mock and negative control samples, respectively. P<0.05 was considered statistically significant using one-tailed paired student's t test. NC; negative control group. These results were not replicated in MCF-7 and MDA-MB-231, indicating that XPA might not be a validated target for miR-145-5p. However, miR-145-5p interaction with XPA mRNAs might not have an effect at the mRNA level, but it may still inhibit protein synthesis. Thus, evaluating the impact of miR-145-5p transfection on XPA protein would determine whether XPA is a confirmed target.

RAD23B expression was not significantly reduced by miR-145-5p transfection in any of the three cell lines (FIG. 10), indication that RAD23B might not be targeted by miR-145-5p. However, protein expression could still be affected by miR-145-5p without seeing an impact on the mRNA level. FIG. 10 is a graph showing RAD23B gene expression regulation by miR-145-5p in MDA-MB-231, MCF-7, and JL BTL-12 breast cancer-derived cell lines. RAD23B gene expression was significantly reduced in miR-145-5p transfected samples compared to mock samples only in the MDA-MB-231 cell line. The fold change in RAD23B gene expression is expressed relative to the mock samples. Error bars represent standard error over five independent experiments for each cell line. + indicates a significant decrease in RAD23B gene expression vs. mock. P<0.05 was considered statistically significant using one-tailed paired student's t test. NC; negative control group.

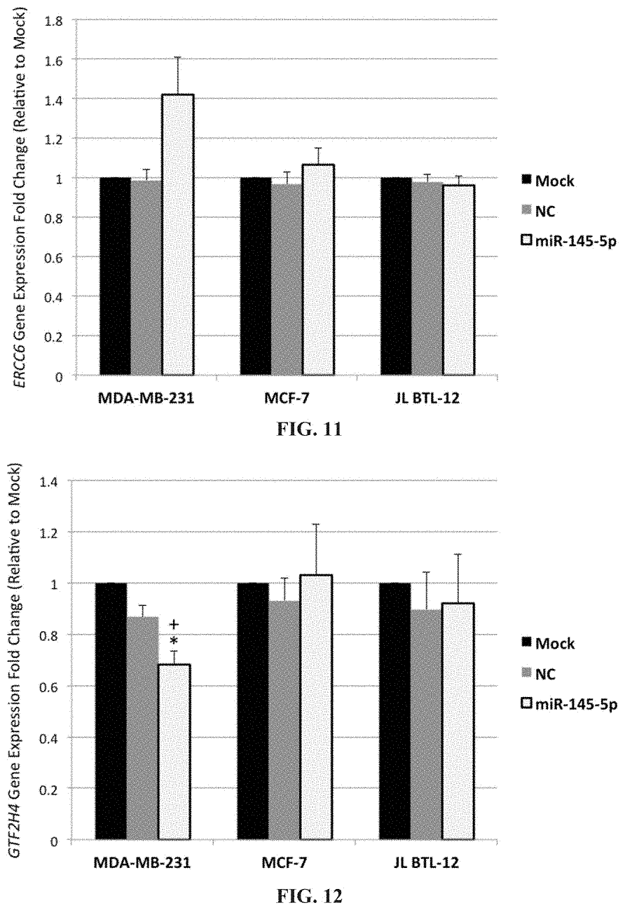

MicroRNA-145-5p was predicted to interact with ERCC6 and GTF2H4 at the promoter region, presumably regulating ERCC6 and GTF2H4 gene expression pre-transcriptionally. However, the steady state mRNA expression of neither of these genes was not significantly affected by the presence of transfected miR-145 compared to mock and negative control in the three cell lines. These results are inconsistent with the proposed promoter-based regulation (FIGS. 11 and 12).

FIG. 11 is a graph showing ERCC6 gene expression regulation by miR-145-5p in MDA-MB-231, MCF-7, and JL BTL-12 breast cancer-derived cell lines. ERCC6 gene expression was not significantly reduced by miR-145-5p transfection in any of the three cell lines. The fold change in ERCC6 gene expression is expressed relative to the mock samples. Error bars represent standard error for five independent experiments for each cell line. P<0.05 was considered statistically significant using one-tailed paired student's t test. NC; negative control group.

FIG. 12 is a graph showing GTF2H4 gene expression regulation by miR-145-5p in MDA-MB-231, MCF-7, and JL BTL-12 breast cancer-derived cell lines. GTF2H4 gene expression was significantly reduced in miR-145-5p transfected samples compared to mock and negative control samples only in the MDA-MB-231 cell line. The fold change in GTF2H4 gene expression is expressed relative to the mock samples. Error bars represent standard errors over five independent experiments for each cell line. + and * indicate significant decreases in GTF2H4 gene expression vs. mock and negative control samples, respectively. P<0.05 was considered statistically significant using one-tailed paired student's t test. NC; negative control group.

The most significantly overexpressed gene, RPA3, in the late stage breast cancer group was predicted as a target for the passenger strand miR-145-3p. RPA3 gene expression was significantly reduced by miR-145-3p transfection in all three of the cell lines tested (FIG. 13). FIG. 13 is a graph showing RPA3 gene expression regulation by miR-145-3p in MDA-MB-231, MCF-7, and JL BTL-12 breast cancer-derived cell lines. RPA3 gene expression was significantly reduced in miR-145-3p transfected samples compared to mock and negative control samples in all three tested cell lines. The fold change in RPA3 gene expression is expressed relative to the mock samples. Error bars represent standard error over five independent experiments for each cell line. + and * indicate significant decreases in RPA3 gene expression vs. mock and negative control samples, respectively. P<0.05 was considered statistically significant using one-tailed paired student's t test. NC; negative control group. Specifically, in MDA-MB-231, RPA3 mRNA was reduced by 34.06% (P<0.001) and 17.63% (P=0.012) in miR-145-3p transfected samples compared to mock and negative control samples, respectively. In MCF-7, it was decreased by 30.34% (P=0.003) and 23.59% (P=0.001) in miR-145-3p transfected samples compared to mock and negative control samples, respectively. In JL BTL-12, miR-145-3p transfected samples had 25.34% (P<0.001) and 18.46% (P=0.001) decreases in RPA3 gene expression relative to mock and negative control samples, respectively. These results suggest that miR-145-3p might play an important role in RPA3 gene regulation by inducing mRNA degradation.

An off-target gene, ERCC1 expression in response to each miR-145 strand was evaluated to see whether miR-145 had an indirect effect on genes that did not have a seed match. Neither miR-145-3p nor miR-145-5p altered ERCC1 gene expression (FIG. 14), suggesting the impact of both strands on function was caused by direct effects on target genes and that they did not extend their impact to affect non-target genes. FIG. 14 is a graph showing ERCC1 gene expression regulation by both strands miR-145-3p and miR-145-5p in MDA-MB-231, MCF-7, and JL BTL-12 breast cancer-derived cell lines. The off-target ERCC1 gene expression was only significantly reduced in miR-145-3p transfected samples compared to mock samples in MDA-MB-231. The fold change in ERCC1 gene expression is expressed relative to the mock samples. Error bars represent standard error over five independent experiments for each cell line. + indicates a significant decrease in ERCC1 gene expression compared to mock samples. P<0.05 was considered statistically significant using one-tailed paired student's t test. NC; negative control group. However, this initial finding should be confirmed by evaluating the impact of both strands on ERCC1 protein expression and to a large extend, by studying a larger set of miR-145 off-target gene and protein expression.

### MirR-145 Target Protein Expression

Besides the effect on gene expression, the impact of miR-145-5p transfection on protein expression of two target genes, XPC and XPA, was assessed in addition to the impact of miR-145-3p on the target RPA3 protein expression. XPC protein expression was significantly suppressed by the leading strand miR-145-5p in all the three cell lines (FIGS. 15A-B). FIG. 15A is a representative Western Blot analysis of XPC protein expression regulating by miR-145-5p in MDA-MB-231, MCF-7, and JL BTL-12 breast cancer-derived cell lines. Representative western analysis of XPC protein in mock, negative control, and miR-145-5p transfected samples from three breast cancer derived cell lines. GAPDH was used to normalize XPC protein expression values. FIG. 15B is a graph showing the fold change of XPC protein expression in MDA-MB-231, MCF-7, and JL BTL-12 breast cancer-derived cell lines. Fold change of XPC protein expression in the three cell lines. XPC protein expression is expressed relative to the mock samples. XPC protein expression was significantly reduced in miR-145-5p transfected samples compared to mock and negative control samples in MDA-MB-231 and MCF-7. Error bars represent standard error for four independent experiments for each cell line. + and * indicate significant decreases in XPC protein expression vs. mock and negative control samples, respectively. P<0.05 was considered statistically significant using one-tailed paired student's t test. NC; negative control samples.

The reduction in MDA-MB-231 miR-145-5p transfected samples was 30.82% (P=0.031) and 32.91% (P=0.048) compared to mock and negative control samples, respectively. In MCF-7, the miR-145-5p transfected samples had 22.85% (P=0.018) and 19.80% (P=0.009) reduction in XPC protein expression compared to mock and negative control samples, respectively. In JL BTL-12, the miR-145-5p transfected samples had 40.57% (P=0.034) and 31.56% (P=0.031) decrease in XPC protein expression compared to mock and negative control samples, respectively. These results confirmed (for the first time) that XPC is a validated target for miR-145-5p.

XPA has emerged as a top candidate for miR-145-5p targeting based on the microRNA target database analyses. Although XPA gene expression was not significantly reduced in MDA-MB-231 and MCF-7, the impact of miR-145-5p might be seen on XPA protein expression by a translation or post-translational mechanism. XPA protein expression was indeed significantly reduced in all three cell lines by miR-145-5p transfection (FIGS. 16A-B). FIG. 16A is a representative Western Blot analysis of XPA protein expression regulating by miR-145-5p in MDA-MB-231, MCF-7, and JL BTL-12 breast cancer-derived cell lines. Representative western analysis of XPA protein in mock, negative control, and miR-145-5p transfected samples from three breast cancer derived cell lines. GAPDH was used to normalize XPA protein expression values. FIG. 16B is a graph showing the fold change of XPA protein expression in MDA-MB-231, MCF-7, and JL BTL-12 breast cancer-derived cell lines. Fold change of XPA protein expression in the three cell lines. XPA protein expression was expressed relative to the mock samples. XPA protein expression was significantly reduced in miR-145-5p transfected samples compared to mock and negative control samples in MDA-MB-231 and MCF-7. Error bars represent standard error for four independent experiments for each cell line. + and * indicate significant decreases in XPA protein expression compared mock and negative control samples, respectively. P<0.05 was considered statistically significant using one-tailed paired student's t test. NC; negative control samples. In MDA-MB-231, miR-145-5p transfected samples had a 29.28% (P=0.007) and 26.44% (P=0.019) reduction compared to mock and negative control samples, respectively. In MCF-7, XPA protein expression was reduced in miR-145-5p transfected samples by 22.90% (P=0.007) and 18.52% (P=0.011) compared to mock and negative control samples, respectively. In JL BTL-12, miR-145-5p transfected samples had a 42.80% (P=0.009) and 33.73% (P=0.011) reduction compared to mock and negative control samples, respectively. These results showed for the first time that XPA expression was regulated by miR-145-5p and suggest that miR-145-5p interacts with XPA mRNA in a way that does not induce XPA transcript degradation but interferes with the protein synthesis process.

The passenger strand miR-145-3p significantly reduced the target RPA3 protein expression in MDA-MB-231 and MCF-7 (FIGS. 17A-B). FIG. 17A is a representative Western Blot analysis of RPA3 protein expression regulating by miR-145-3p in MDA-MB-231, MCF-7, and JL BTL-12 breast cancer-derived cell lines. Representative western analyses of RPA3 protein in mock, negative control, and miR-145-3p transfected samples from three breast cancer derived cell lines. GAPDH was used to normalize RPA3 protein expression values. FIG. 17B is a graph showing the fold change of RPA3 protein expression in MDA-MB-231, MCF-7, and JL BTL-12 breast cancer-derived cell lines. Fold change in RPA3 protein expression in the three cell lines. RPA3 protein expression was expressed relative to the mock samples. RPA3 protein expression was significantly reduced in miR-145-3p transfected samples compared to mock and negative control samples in MDA-MB-231 and MCF-7. Error bars represent standard errors for four independent experiments for each cell line. + and * indicate significant decreases in RPA3 protein expression compared mock and negative control samples, respectively. P<0.05 considered statistically significant using one-tailed paired student's t test. NC; negative control samples 3p; miR-145-3p transfected samples. The reduction in MDA-MB-231 miR-145-3p transfected samples was 21.07% (P=0.042) and 23.28% (P=0.024) compared to mock and negative control samples, respectively. MCF-7 miR-145-3p transfected samples had 15.12% (P=0.045) and 21.18% (P=0.018) decrease in RPA3 protein expression compared to mock and negative control samples. These results indicated for the first time that the passenger strand miR-145-3p has a functional role in NER regulation through modulation of RPA3 gene and protein expression in MDA-MB-231 and MCF-7.

XPC and XPA Putative miR-145 Binding Sites are Targeted by miR-145

The significant decrease of miR-145 in late stages of breast cancer cell lines and its inhibitory effect on the protein expression of XPC and XPA prompted the instant inventor to investigate whether miR-145 binds the putative binding site of these genes. In order to investigate the interaction between miR-145 and XPA and XPC, the dual luciferase reporter construct (commercially-available pEZX-MT06; FIG. 18, Genecopia) which contains a firefly luciferase gene subcloned to include the miR145 target sequence (T) from either XPC or XPA were created. In each target vector the binding sites were placed into the 3′ untranslated region of the firefly luciferase gene. These vectors also contained a renilla luciferase gene to control for transfection efficiency. Mutated versions of these target sequences (M) were also created that would not interact with miR145 as negative standards of comparison.

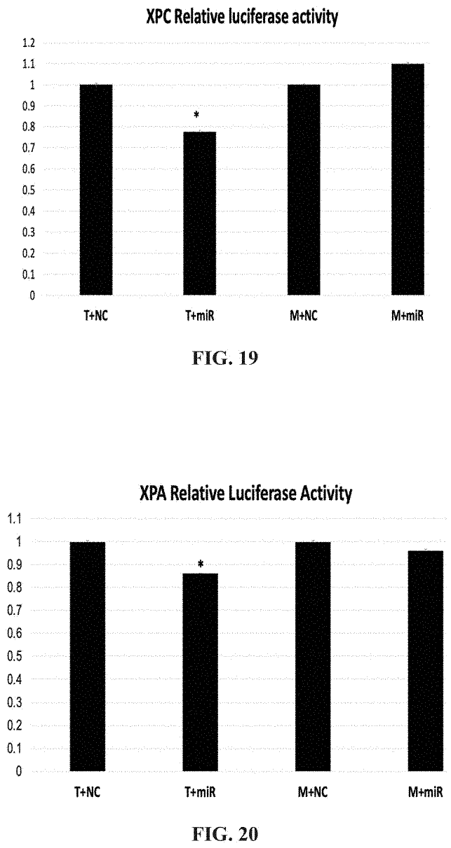

As shown in FIGS. 19 and 20, the commercially generated dual luciferase vectors (Genecopia) were used to measure relative luciferase activity for putative miR145 binding sites or mutated versions of each binding site that would be incapable of binding to miR145. The binding site of interest for XPC or XPA was subcloned into the 3′ UTR of the firefly luciferase vector and transfected in the presence of miR145 or a negative control scrambled RNA (Ambion). Firefly luciferase was measured relative to renilla luciferase from the same vector to produce a ratio that controlled for transfection efficiency.

Using Lipofectamine (3000) MDA MB-231 cells were co-transfected with plasmid plus miR145 (15 nM) or negative control scrambled RNA. Vectors contained the target site (T) or mutant version (M) of the miR145 binding site. For XPC, 150 ng of plasmid was co-transfected with 15 nM miR145. Firefly luciferase signal was divided by the Renilla signal, giving the relative luciferase ratio for each gene. FIG. 19; N=2 experiments, P=0.01, T+target site vector, M=mutant site vector, miR=miR-145; and NC=negative control (scrambled RNA). For XPA 75 ng of plasmid was co-transfected with miR145 (15 nM) or NC. Luciferase signal was divided by the Renilla signal, giving the relative luciferase ratio for each gene. FIG. 20; N=4 experiments, P=0.012, T+target site vector, M=mutant site vector, miR=miR-145; and NC=negative control (scrambled RNA).

Each vector was co-transfected into (160,000) MDA MB-231 cells with miR145 (15 nM) or negative control scrambled RNA (Ambion). MDA MB231 cells represent stage IV breast cancer and have been used throughout this study as one of three cell lines representing late stage breast cancer. Luciferase assays showed the miRRA mutant vectors for XPC and XPC did not significantly decrease the relative firefly luciferase expression relative to renilla luciferase.

The ratio of firefly luciferase reporter activity relative to renilla luciferase activity for the target binding site (T) in the presence of miRRA showed significantly lower relative luciferase activity than the target binding site in the presence of the negative control (scrambled RNA) for both XPC (decreased by 23%) and XPA (decreased by 15%) (FIGS. 19 and 20). The mutated binding site for each gene was also placed into the same vector and showed no significant difference between the miR145 transfected and negative control transfected cells.

### Experimental Study: Part 2 (Includes Reference List)

As noted, treatment resistance is the central problem of all cancer therapeutics and accounts for an estimated 90% of deaths of cancer patients (1,2). Despite all the different types of treatments for breast cancer (BC), an advanced or recurrent tumor can be or become resistant to any of them. Using the GEO database and tumor-derived BC explants/cell lines of the instant inventor, it has been shown that advanced BCs have significantly higher functional nucleotide excision repair (NER) and gene expression than stage I BC. This high NER capacity is a major factor in genotoxic drug resistance. A major obstacle in the field of BC research is the lack of understanding of the engine of drug resistance, i.e., the genomic instability intrinsic in the tumor, rather than a specific mechanism tied to the action of a specific class of drugs. Genomic instability is a primary engine of treatment resistance because the mutations in tumors drive all kinds of treatment resistance. There are five DNA repair pathways that minimize genomic instability in humans. As discussed previously, based on data disclosed herein and the literature, NER plays a role in multiple sporadic cancers, including breast, bladder and testicular cancer (3-5). By using a panel of BC cell lines created from 60 patients representing different stages and subtypes, the instant inventor has discovered a microRNA (designated as miRRA) that binds to three of the 20 canonical genes in the Nucleotide Excision Repair (NER) pathway, effectively lowering protein translation from these respective mRNAs (XPA, XPC and RPA3) and significantly lowering functional NER. This has been verified by direct binding studies of miRRA on target regions of these genes and testing the impact of cisplatin, which causes intra-strand crosslinks remediated by NER, with and without miRRA. miRRA enhancement of cisplatin cytotoxicity in three advanced BC cell lines occurred dose dependently in experiments described herein. MiRRA is naturally present in non-diseased heart, kidney, liver, and breast tissues.

In order to address the gap in knowledge about the engine of drug resistance, several cell lines were chosen that have escaped different types of therapies. It has been shown that MDA MB231, a triple negative BC (TNBC), stage IV chemotherapy-resistant cell line, has high functional NER and high expression of NER genes. It has been shown that NER in this cell line is significantly decreased in the presence of miRRA, and that it is more susceptible to cisplatin cytotoxicity in the presence of miRRA. The hypothesis of the instant inventor is that miRRA will effectively lower NER capacity in advanced/aggressive BC, allowing for the application or reapplication of chemotherapy that will be significantly more effective after pretreatment with miRRA. The long-term objective (of the instant inventor) is to validate that this novel microRNA therapeutic will reduce NER and allow for a second chance at control or cure of advanced stage BC. MCF7 a stage IV, hormone therapy-resistant luminal type BC has high functional NER and high canonical gene expression of NER genes. MCF7 was similarly impacted by miRRA-enhanced cisplatin cytotoxicity. Luminal BCs represent ⅔ of American BCs and recur 50% of the time in patients after becoming resistant to hormone-based treatments. A second luminal cell line has also been studied that was chemotherapy naïve (JL BTL-12), which showed similar results to MCF7, indicating that aggressive BC does not need to undergo the gauntlet of therapy to possess high NER capacity. That said, TNBCs seem to have the highest relative levels of NER. The instant inventor therefore hypothesizes that specific BC types may show preferential enhancement of miRRA cytotoxicity in drug treatment studies. The objectives of the experimental studies described herein:

Objective 1. Analyze a total of 5 TNBC cell lines, establishing the repair capacity using the functional UDS assay, NER gene expression using RNA sequencing and determining the cytotoxicity of cisplatin and Adriamycin, each in combination with miRRA on MDA MB231 and these additional 4 cell lines. Adriamycin is an agent specifically used treating in TNBC and causes damage consistent with NER remediation.
Objective 2. Analyze a total of 5 luminal type BC cell lines establishing the functional repair capacity using the UDS assay, the NER gene expression using RNA sequencing and determining the cytotoxicity of cisplatin and Adriamycin, each in combination with miRRA on these additional 3 cell lines. MCF7 and JL BTL-12 have been characterized.
Objective 3. Establish the efficacy of miRRA as an in vivo therapeutic in mouse tumor xenografts by selecting 3 of each type of cell line mentioned above, i.e., those most efficient at producing tumors, and encompassing 3 TNBC and 3 Luminal BC. Once tumors have been established, injections of cisplatin or Adriamycin will be performed with and without miRRA pretreatment, and measurements of tumor diameter will be made.

The instant inventor has shown that NER is epigenetically controlled by at least one microRNA (miRRA) and (the instant inventor) is the first to establish translational therapeutic control of any DNA repair pathway. It is expected that the additional cell lines analyzed in this study will confirm what was seen in the foundational work (discussed above-Part 1), although certain subtypes may be optimal for the use of miRRA. It is expected that pretreatment with miRRA will lead to enhanced tumor shrinkage with chemotherapy drugs in xenograft mouse models as opposed to chemotherapy alone. miRRA binds to multiple genes in the NER pathway and therefore represents a powerful strategy for lowering drug resistance through modulation of DNA repair. This work may allow for a second chance at cure or control in late-stage BC and may also allow for therapeutic repurposing of drugs.

Significance of this Research

Recurrent/metastatic breast cancer (BC) almost always has a lethal outcome. Treatment resistance is the central problem of all recurrent or advanced stage cancers. Genomic instability is a primary engine of treatment resistance. There are five DNA repair pathways that minimize genomic instability in humans. As discussed previously, nucleotide excision repair (NER) plays a role in multiple sporadic cancers, including breast, bladder and testicular cancer (3-5). The instant inventor is the first to show that NER capacity is correlated with increasing stage in sporadic BC. What is critically needed is the development of drugs that decrease treatment resistance, since no matter what class of drug is used, the tumors eventually develop resistance. Using a continuum of BC cell lines created from over 60 patient tumor samples, the instant inventor has discovered a microRNA, designated miRRA, that directly impacts the function of NER in BC cell lines. Using cisplatin, a classic DNA crosslinking agent remediated by NER, it has been shown that increased cytotoxicity in the presence of miRRA occurs in the MCF7, MDA MB231 and JL BTL12 cell lines. Because of the instant inventor's work in BC, the potential of miRRA to increase the efficacy of genotoxic chemotherapy in late-stage BC can be validated. Cisplatin, as well as Adriamycin, will be used. Adriamycin is a frontline BC agent for Triple Negative BC (TNBC) that does not require liver metabolism to be active and intercalates into DNA, affecting the geometry of the DNA helix such that it is detected and remediated by NER. BC is not a monolithic disease. Two major types of BC will be examined in this experimental study, TNBC and luminal type. The transformational part of this research is that the engine of resistance itself is being attacked, by downregulating DNA (Nucleotide Excision) Repair with a microRNA that targets three NER genes.

Innovation of this Research

Basic Science Innovation: The ability of the laboratory of the instant inventor to establish cell lines from patient samples with an 85% success rate that represent stage 0-IV and all subtypes of breast cancer (BC) has allowed for identification of miRRA as a naturally occurring microRNA present in non-diseased breast tissue and stage I BC, but as absent in stage IV BC cell lines (both laboratory-derived and commercially available stage IV cell lines) (3, 6-10). The laboratory's ability to culture these patient tumors as primary cultures to perform the functional Unscheduled DNA Synthesis (UDS) assay for nucleotide excision repair (NER) allowed for the instant inventor to ascertain that tumor stage correlated significantly with NER function. Very few basic science studies on BC discriminate patient samples by stage, including studies on NER and BC (11). It is the belief of the instant inventor that a great deal of information about treatment resistance has been missed by this lack of stage-specific discrimination in the literature.

Multiple classes of drugs have been developed for BC. For all classes of treatment, drug resistance is inevitable. The instant experimental study is targeting the foundation of drug resistance, genomic instability. Genomic instability is caused by the loss of DNA repair; in fact, most of the seminal tumor suppressor genes are DNA repair related. NER is a pathway that only recently has been identified as altered in sporadic, non-familial BC, and the instant inventor's laboratory was the first to publish on this subject (3).

MicroRNA drugs have not yet been used clinically for BC as part of standard of care regimens. The microRNA identified by the instant inventor interacts with a minimum of 3 NER gene transcripts and as many as 12. Direct binding with NER gene transcripts XPA, XPC and RPA3 has been shown. Bladder, testicular and ovarian cancer have also been identified as having elevated NER function and may show benefit from this microRNA as well. In the past year, in vitro mRNA binding studies and cytotoxicity studies using cisplatin have been performed. miRRA increased the efficacy of cisplatin on three BC cell lines representing TNBC and luminal type, also representing previously drug treated BC and chemotherapy naïve stage III BC. This means that advanced disease, whether representing intrinsically or acquired drug resistance, is vulnerable to miRRA.

Clinical Innovation: Resistance to certain chemotherapy drugs, such as cisplatin, has been linked to an increase in DNA repair, specifically NER. Cancer cells can induce NER, via overexpression of NER genes, leading to drug resistance. Research in the lab of the instant inventor shows the clinical importance of the NER pathway that could be used as a predictor of treatment resistance (12). The innovative approach (of the invention) is to attack the engine of resistance, NER, by using miRRA to sensitize late-stage BC cells to existing chemotherapy drugs like cisplatin and Adriamycin. This could be a huge benefit to those with late-stage BC. Instead of developing new genotoxic drugs, clinicians could use a combination of miRRA plus a known and repurposed chemotherapy drug and potentially increase the time late-stage BC patients are in remission.

Since Adriamycin is an existing BC treatment and it is often used in combination with other chemotherapy drugs (cyclophosphamide, paclitaxel, 5-flourourocil), determining its increased cytotoxicity when combined with miRRA could be a great benefit for BC patients. It is used in the treatment of both early TNBC and late-stage BC, as well as metastatic disease. Adriamycin is known as the “Red Devil” because of its severe side effects and its red appearance. It is cardiotoxic, which is dose related. If the addition of miRRA can decrease the amount of Adriamycin needed to treat or decrease the number of cycles of the drug, then it would indeed be beneficial to the patient, hopefully reducing the probability of dying from heart failure due to their BC treatment. Since miRRA is present in healthy tissues (heart, liver, kidney and breast), the possible side effects should be minimized and not add additional side effects to the chemotherapy treatment, as the instant inventor hypothesizes that there would be no additional effects related to overexpression.

### Data

Breast cancer (BC) is the most diagnosed cancer among women in the United States. In 2021, there are predicted to be 281,550 new cases of invasive BC (in the United States). There is not one specific cause of BC, as both genetic makeup and environment can influence the risk of developing BC. There are five molecular subtypes of BC which are treated differently in the clinic.

BC Staging: Currently, BC is staged 0-IV and the staging is based on the tumor-node metastasis system. The size of the tumor, lymph node status and metastasis are evaluated to determine BC stage (13). Stage IV BC has a 10-year survival rate of 13.3% compared to that of stages II and III BC at 66.5% (14), a dramatic decrease. This reduced survival in stage IV disease shows the importance of investigating treatments that could be used for late-stage BC that has metastasized to distant areas of the body. TNBC is, stage for stage, one of the most lethal forms of BC (15). At the present time the evidence about DNA repair capacity in TNBC is controversial (16,17).

BC Treatment: Today, treatment of BC can involve chemotherapy, radiation therapy, surgery, hormone therapy, small molecule inhibitor treatment and monoclonal antibody treatment (with and without a toxic payload attached to the monoclonal antibody). When detected early, a lumpectomy is performed, which is followed with radiation therapy to kill any remaining cancerous cells. At later stages chemotherapy is needed and can include Adriamycin, Taxol, 5-fluorouracil, cyclophosphamide and carboplatin, and cisplatin. Most treatment plans for early BC include a combination of chemotherapy or biologic drugs so that doctors can attack the tumor in multiple ways and by doing so, reduce clonal selection of drug resistant tumor cells. Chemotherapy drug resistance accounts for roughly 90% of deaths of cancer patients (1,2) when death does not occur from other causes (often related to the side effects of chemotherapy). The use of genotoxic chemotherapy agents can often lead to treatment resistance in cancer cells, as they can result in upregulated DNA repair pathways in response to DNA damage.

DNA Repair: Loss of DNA repair can lead to the accumulation of mutations over time followed by malignancy. Most researchers place a priority in BC on double strand break repair, as it is associated with the inherited BRCA1 and BRCA2 genes. In contrast, NER plays a role in remediating DNA damage due to geometric alterations in the DNA helix (i.e., cyclobutene pyrimidine dimers, 6-4 photoproducts, DNA crosslinks, bulky adducts, and intercalation) formed by multiple chemotherapeutic drugs. Mutations in the NER repair pathway can lead to cancer and cancer-prone syndromes, such as xeroderma pigmentosum (XP), Lynch syndrome and Bloom's syndrome (18). Patients with XP are also at increased risk of developing internal cancers such as BC and lung cancer (19). NER had not been widely studied in sporadic BC until the instant inventor published (3), but shortly afterward Matta et al. (11) published a correlation between NER and estrogen receptor status in the blood of BC patients using the host cell reactivation (HCR) assay to measure transcription-coupled repair in actively transcribed genes (represents 3% of global genomic repair). Their work is significant due to the prevalence of ER+BC cases that are diagnosed, but BC stage was not taken into consideration in their study. The instant inventor studies global genomic NER, which scans and repairs the entire human genome, on tumor cells.

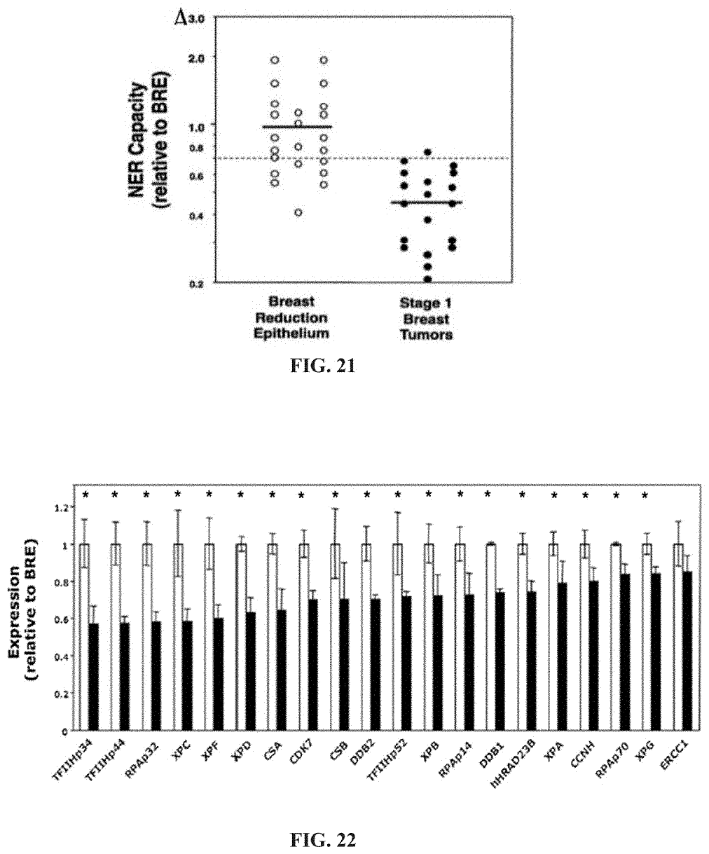

NER in BC: As shown using the functional UDS assay, nucleotide excision repair (NER) capacity is decreased in early-stage BC compared to breast reduction epithelium (BRE) (FIG. 21) (3). NER capacity of stage I primary cultures is significantly lower than non-diseased BRE. Each dot (FIG. 21) represents a single patient's non-diseased BRE from mammoplasty (left) versus stage I breast cancer (right).

Gene expression microarray, validated by RNase protection, confirmed a decrease in gene expression in 19 out of 20 canonical NER genes (FIG. 22). The gene expression microarray shows that 19 out of 20 canonical NER genes are significantly downregulated in 3 stage I tumors (black bars) compared to 3 BRE tissue samples (white bars). The loss of functional DNA repair correlated with a decrease in both mRNA (FIG. 22) and protein expression (protein data not shown).

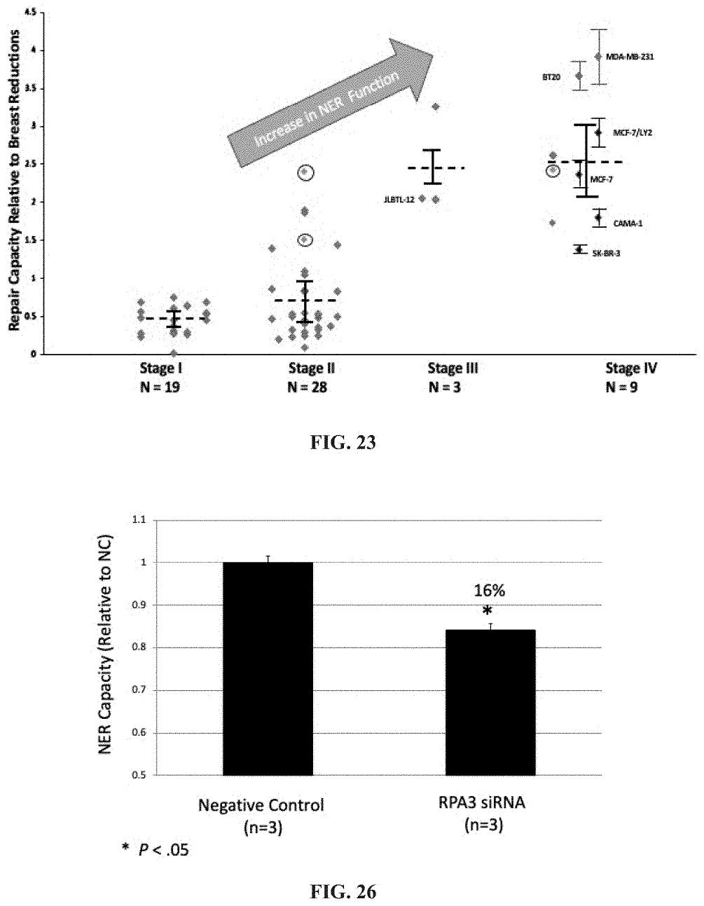

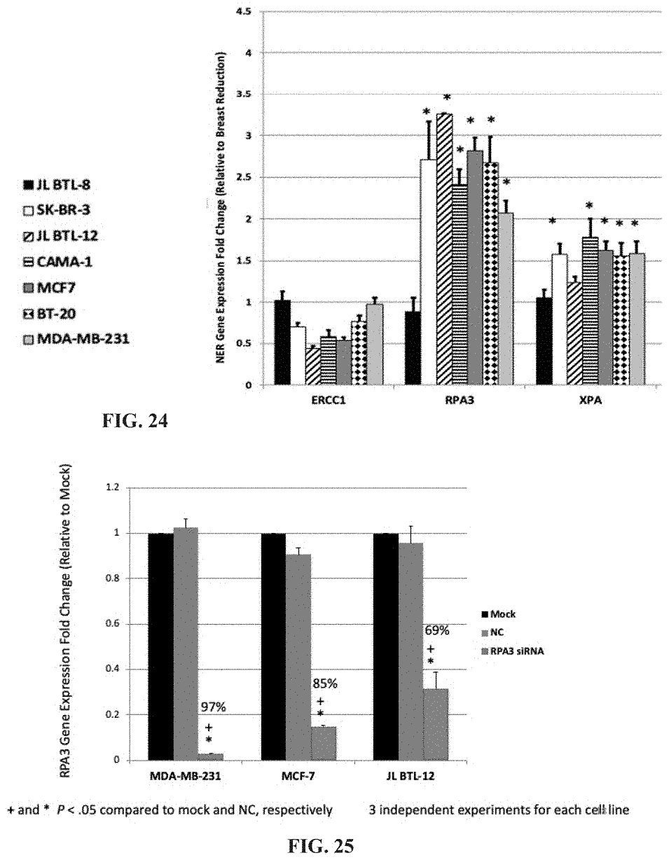

NER capacity (function) increases as stage increases (progresses) from stages I-IV (FIG. 23). Primary cultures from women with various stage I-IV breast tumors and some commercially available cell lines (Stage IV column) were analyzed for NER capacity using the functional UDS assay. This functional assay is used to diagnose rare NER deficiency syndromes like xeroderma pigmentosum (XP) (predisposed to skin and other internal cancers, including BC) (19-21). Gene expression data confirmed that 12/20 of the canonical NER genes were significantly increased in expression between stage I and stage IV breast cancer. Cell lines are defined as using >13 passages and explants <13 passages. Selected genes are shown in FIG. 24. The graph of FIG. 24 shows differences in gene expression between stage I breast cancer (JL BTL-8 breast cancer-derived cell line) and late-stage breast cancer (SKBR3, JL BTL-12 (stage III), Cama-1, MCF7, BT-20, and MDA MB231 breast cancer-derived cell lines). ERCC1 does not show significant differences in expression between early and late-stage BC. These differences were determined using microarray and independently validated using RT-PCR. All data shown are relative to one internal standard BTL cell line that represents the midpoint of the breast reduction mammoplasties. * Asterisks represent significant differences between stage I and late-stage tumor lines.

Nucleotide excision repair (NER) is dysregulated in breast cancer. Epigenetic regulation of NER occurs in the progression of breast cancer. An epigenetic loss in NER from non-diseased breast to stage I followed by a gain of NER with tumor progression is illustrated in FIG. 3. This loss is not due to methylation of the NER genes, promoter regions or CpG islands (data not shown) and thus, the instant inventor considered microRNAs as possible mediators of this epigenetic effect.

The specific role of RPA3 in functional NER can be seen in FIGS. 25 and 26 based on siRNA experiments followed by performing the Unscheduled DNA Synthesis (UDS) assay after transfection with the siRNA vector that effectively decreases RPA3 mRNA by 97% in MDA MB231 cells. Global NER function is decreased by 16% which indicates that other genes that presumably include XPA and XPC also play a role. Specifically, FIG. 25 shows that RPA3 gene expression was significantly reduced in RPA3 siRNA samples compared to mock and NC samples. RPA3 siRNA successfully silenced RPA3 gene expression in three advanced stage BC cell lines. + and * P<0.05 compared to mock and NC, respectively. Three independent experiments were conducted for each cell line. Specifically, FIG. 26 shows that silencing of the RPA3 gene expression in MDA MB 231 cells significantly reduced NER function. * P<0.05

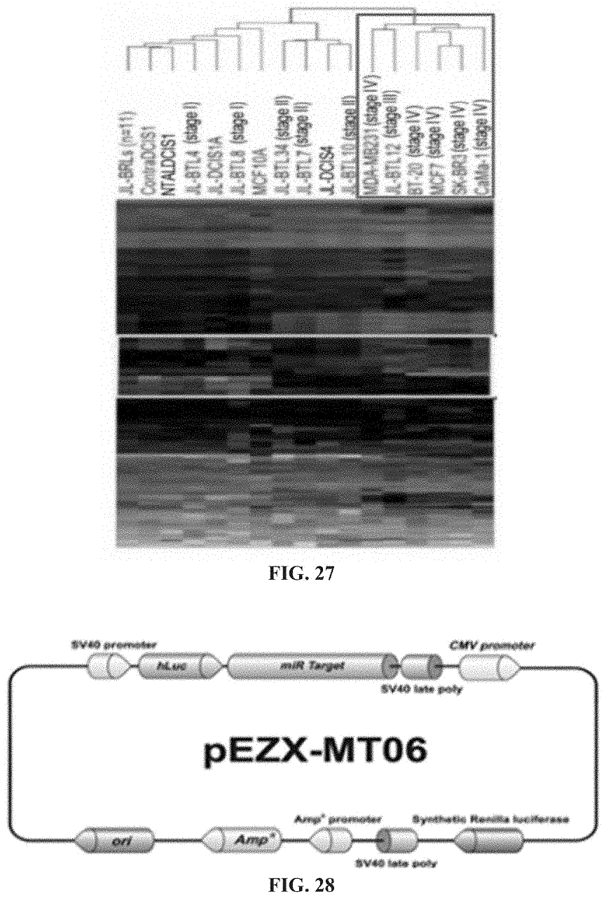

Microarray and RNAseq analysis of replication/DNA repair genes also provided a distinct gene expression profile of late-stage BC cell lines compared to early-stage cell lines (FIG. 27) with laboratory cell lines conforming to patterns shown by commercially available cell lines of the same stage. In FIG. 27, hierarchical clustering, supervised analysis of 521 probe sets covering replication and DNA repair genes based on expression microarray and validated with RNAseq. 3 major clusters are evident: 1. Right: the commercial stage IV pleural effusion lines (MDA MB231, MCF7, SKBR3, etc.) and laboratory stage III cell line JL BTL-12. 2. Middle cluster: stage II cell lines/explants created in the laboratory of the instant inventor; 3. Left: stage I cell lines, DCIS cell lines with matching contralateral and ipsilateral non tumor adjacent explants as well as Breast Reduction Lines (BRLs). MCF10A clusters with laboratory early stage or non-diseased (BRL) group of explants/cell lines are also shown.

miRRA in Relation to Breast cancer (BC): As previously noted, MicroRNAs (miRNAs) are a class of small, endogenous inhibitory noncoding RNAs. They are about 20-25 nucleotides in length and are responsible for the regulation of gene expression post-transcriptionally by binding to the complementary sequence on mRNAs. Researchers have found that miRNAs can cause a change in expression levels across different types of cancer and are involved in regulating DNA damage and repair genes (22). MiRNAs have been studied in BC and play a role in initiation, progression, and metastasis, as they can affect apoptosis, drug targets, transport and metabolism (23). Many studies have linked miRNAs to chemotherapy resistance in cancers, including BC (24-26). Xie et al. (27) demonstrated that miRNA dysregulation impacts NER function. They showed that the reintroduction of miR-192 in HepG2, which is a well-known hepatocellular carcinoma cell line, significantly reduced NER function by suppressing the NER genes ERCC3 and ERCC4 and their protein expression. However, this research group used the HCR assay to measure the functional impact of miR-192 transfection on NER function. HCR measures transcriptional-coupled NER repair, which represents a small percentage (3%) of the total NER repair and might not be truly representative of overall NER function. Several groups, such as Cataldo et al. (28) have shown that miRNAs can sensitize cancer cells to DNA-damaging drugs, such as those used in chemotherapy. Additionally, Szalat et al. (29) demonstrated that NER inhibition, particularly of XPB (ERCC3), increases sensitivity to alkylating agents in multiple myeloma. He et al. (30) showed that miRNAs, particularly miR-145, sensitizes breast cancer to doxorubicin by targeting and inhibiting the multidrug resistance-associated protein 1 (MRP1).

The laboratory of the instant inventor is the first to demonstrate that the microRNA, “miRRA”, is present in the early stages of breast cancer but absent in late-stage breast cancer (FIGS. 4 and 5). FIG. 4 shows the leading strand miR-145-5p expression in the late-stage breast tumor tissue group compared to the stage I breast tumor tissue group using the Nanostring microRNA expression panel. FIG. 5 shows the passenger strand miR-145-3p expression in the late-stage breast tumor tissue group compared to the stage I breast tumor tissue group using microRNA RT-PCR. miRRA is almost undetectable in late-stage breast cancer. miRRA-5p (left) p=0.0002, and miRRA-3p (right) are low or absent in late-stage BC cell lines using Nanostring and RT-PCR for detection.

Further, it was found that miRRA reduces NER mRNA expression levels in three cell lines: MCF-7, MDA MB-231 and JL BTL-12 (FIGS. 9, 8, 13, 14, and 30 (Table 5)). XPA (FIG. 9), XPC (FIG. 8), RPA3 (FIG. 13) and ERCC1 (FIG. 14) gene expression regulation by miRRA in MDA-MB-231, MCF-7, and JL BTL-12 is shown. Specifically, FIG. 8 shows XPC gene expression regulation by miR-145-5p in MDA-MB-231, MCF-7, and JL BTL-12 breast cancer-derived cell lines. FIG. 9 shows XPA gene expression regulation by miR-145-5p in MDA-MB-231, MCF-7, and JL BTL-12 breast cancer-derived cell lines. FIG. 13 shows RPA3 gene expression regulation by miR-145-3p in MDA-MB-231, MCF-7, and JL BTL-12 breast cancer-derived cell lines. FIG. 14 shows ERCC1 gene expression regulation by both strands miR-145-3p and miR-145-5p in MDA-MB-231, MCF-7, and JL BTL-12 breast cancer-derived cell lines.

XPA, XPC gene expression is significantly reduced in miRRA-5p transfected samples compared to mock and negative control samples in the three cell lines. The fold change in XPC gene expression is expressed relative to the mock samples. Error bars represent standard error for 5 independent experiments for each cell line. + and * indicate significance. RPA3 was significantly downregulated in the presence of miRRA-3p. P<0.05 was considered statistically significant using one-tailed paired student's t-test. NC; negative control group. ERCC1 is a negative control gene that does not have a miRRA-3p or -5p binding site and thus is not affected by miRRA. These data were validated independently with RNA sequencing. Average transfection efficiencies with miRRA for MDA MB231 97%, MCF7 86%, JL BTL-12 74%

FIG. 30 shows Table 5, which summarizes miRRA-3p and miRRA-5p data. XPA does not show changes in mRNA in the presence of miRRA but does show changes in protein as predicted by it 3′UTR binding site. RPA3 and XPC show reductions in mRNA in the presence of miRRA due to their binding sites being present in the 3′UTR and coding sequence, respectively. All three genes show reductions in their protein levels which satisfies the criteria for action of a microRNA. Both strands of miRRA appear to be active depending on the gene of interest.

MiRRA reduces protein expression in XPA, XPC and RPA3 (FIGS. 16A-B, 15A-B, 17A-B, and 30 (Table 5)). Representative western blot analysis showing protein expression of XPA, XPC and RPA3 after miRRA transfection in three breast cancer cell lines. Specifically, FIG. 16A is a representative Western Blot analysis of XPA protein expression regulating by miR-145-5p in MDA-MB-231, MCF-7, and JL BTL-12 breast cancer-derived cell lines. FIG. 16B is a graph showing the fold change of XPA protein expression in MDA-MB-231, MCF-7, and JL BTL-12 breast cancer-derived cell lines. FIG. 15A is a representative Western Blot analysis of XPC protein expression regulating by miR-145-5p in MDA-MB-231, MCF-7, and JL BTL-12 breast cancer-derived cell lines. FIG. 15B is a graph showing the fold change of XPC protein expression in MDA-MB-231, MCF-7, and JL BTL-12 breast cancer-derived cell lines. FIG. 17A is a representative Western Blot analysis of RPA3 protein expression regulating by miR-145-3p in MDA-MB-231, MCF-7, and JL BTL-12 breast cancer-derived cell lines. FIG. 17B is a graph showing the fold change of RPA3 protein expression in MDA-MB-231, MCF-7, and JL BTL-12 breast cancer-derived cell lines.

The level of XPA and XPC protein expression was reduced in miRRA-5p transfected samples compared to mock and negative control samples. miRRA-3p transfected samples also showed a significant decrease in RPA3 protein expression. GAPDH was used to normalize each protein expression value. Error bars represent standard error for 4 independent experiments for each cell line. + and * indicate significant decreases for each protein expression vs. mock and negative control (NC) samples, respectively. P<0.05 was statistically significant using one tailed paired student's t-test. JL BTL-12 was originally grown om Matrigel which lowered the transfection efficiency. When re-transfected on plastic, the transfection efficiency was 86%. Transfection efficiencies: MDA MB231 97% and MCF-7 86%.

It has been shown unequivocally that the presence of miRRA significantly reduces functional NER (FIG. 6). NER function is significantly reduced by both strands of miRRA when compared to the scrambled RNA negative control (NC). FIG. 6 shows the impact of miR-145 on nucleotide excision repair (NER) capacity in MDA-MB-231, MCF-7, and JL BTL-12 breast cancer-derived cell lines. The impact of both miRRA strands after transfection on NER capacity in the three late-stage BC cell lines was investigated using the functional UDS assay. This UDS assay was run on miRRA transfected cells to evaluate functional DNA repair. Average transfection efficiencies with miRRA for MDA MN231 97%, MCF-7 86%, and JL BTL-12 74%. * P>0.05 compared to negative control (NC). NER function was reduced significantly after both the leading strand miRRA5p and the passenger strand miRRA3p were transfected, compared to negative control (NC) transfected cells in all the three cell lines. In MDA-MB-231, NER function was significantly reduced by 7.2% (n=506, P<0.001) and 37.5% (n=420, P<0.001) in miRRA3p and miRRA5p transfected cells, respectively, when compared to NC transfected cells (n=402). MCF-7 miRRA3p and miRRA5p transfected cells showed significantly decreased NER capacity by 27.6% (n=300, P<0.001) and 48.4% (n=300, P<0.001) compared to negative control (NC) treated cells (n=300), respectively. Lastly, JL BTL-12 miRRA3p and miRRA5p transfected cells showed a significant reduction in NER function by 15.6% (n=300, P<0.001) and 41.3% (n=300, P<0.001) in relative to NC transfected samples (n=300).

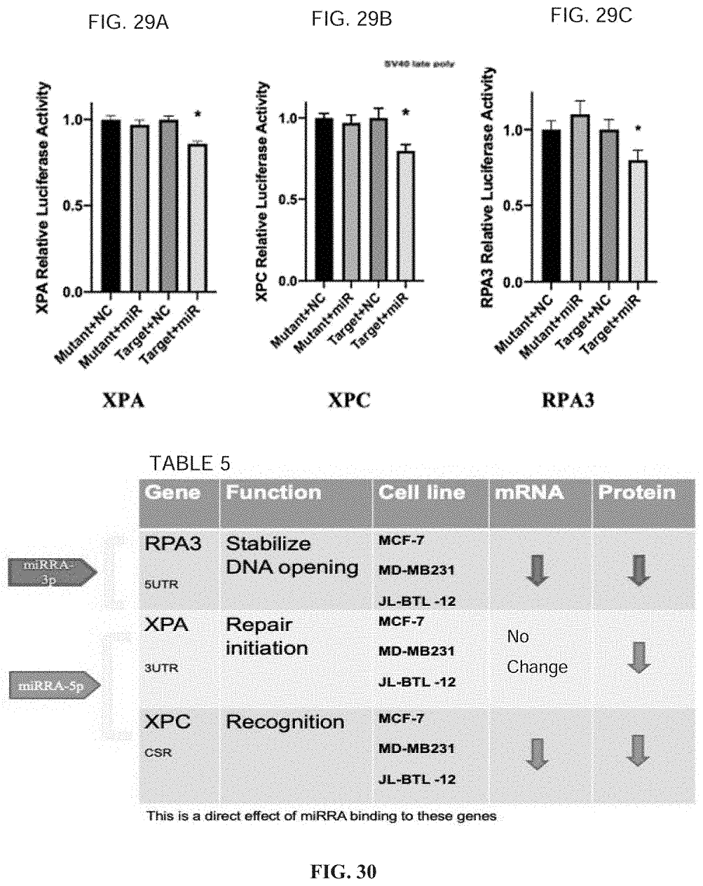

MiRRA binds the predicted target sites of XPA, XPC and RPA3, respectively, not a mutant site, as shown in direct binding studies of the putative binding regions of each of these genes subcloned into a dual luciferase vector shown in FIGS. 28 and 29A-C. Relative luciferase activity is shown, after co-transfecting a dual luciferase vector (FIG. 28) containing the putative miRRA binding site or mutated binding site of XPA, XPC and RPA3 genes, respectively with miRRA 3p or 5p vs negative control (NC). Experiments consisted of miRRA with target site, NC with target site, miRRA with mutant site, NC with mutant site. Either the 3p or 5p miRRA was transfected based on the predicted binding in the target site. The luciferase signal was divided by the Renilla signal, giving the relative luciferase ratio for each gene. Only the target site-containing vector transfected with miRRA, showed a significantly lower luciferase activity indicating specific binding. Error bars represent standard error for 4 independent experiments. Either the mutant sequence or the miRRA target sequence were subcloned into the mIR target site in the pEZX-MT06 vector (FIG. 28). miRRA-3p bound to the target sequence of RPA3 and miRRA-5p bound the target sequence of XPA and XPC. Transfection efficiency with the pEZX-MT06 vector was 71% for these experiments all performed in MDA MB231. pEZX-MT06 vector was 7.5 kb (FIG. 28). Mutant regions which were the reverse complement of each target region were also evaluated for comparison, and all binding was evaluated relative to the same NC RNA. Specific binding to the miRRA target area of the pEZX vector (FIG. 28) caused lowered expression of the luciferase gene relative to the renilla luciferase gene on the same vector. The ratio of expression of the 2 luciferase genes is shown. FIG. 29A is a graph showing XPA relative luciferase activity. FIG. 29B is a graph showing XPC relative luciferase activity. FIG. 29C is a graph showing RPA3 relative luciferase activity. Reduction of protein expression of specific NER genes has been shown to be render tumor cells more sensitive to adriamycin treatment Saffi et al. (31).

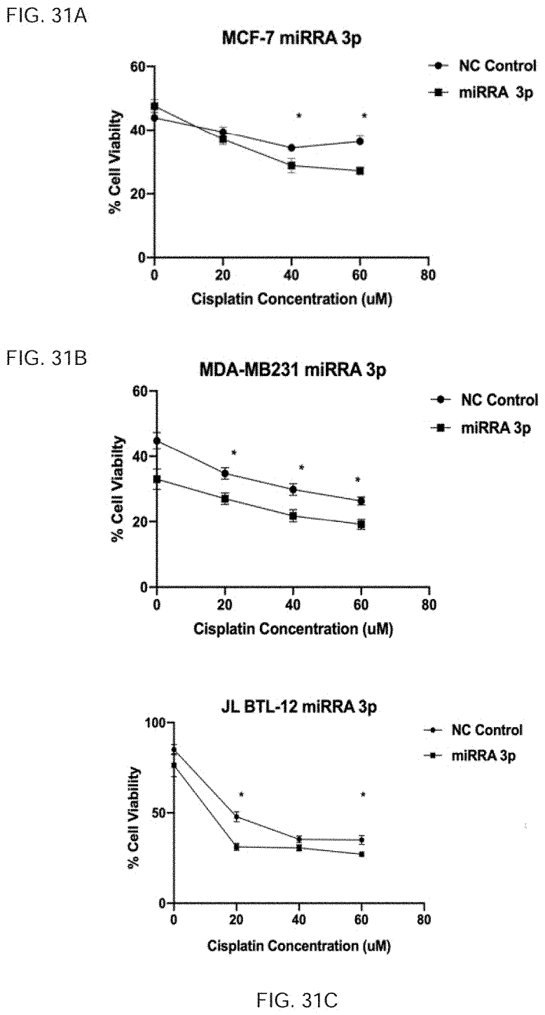

In addition to showing the functional loss of NER in the presence of miRRA and the unequivocal binding data, the clinical significance of these findings is shown in FIGS. 31A-C. miRRA enhances cytotoxicity of cisplatin in three stage IV cell lines.

FIG. 31A shows in vitro cytotoxicity studies using MCF7 luminal type breast cancer (BC) cell line transfected using lipofectamine with miRRA 3-p vs. scrambled RNA negative control (NC) (N=4) in the presence of cisplatin*. Transfection efficiency was 80%.

FIG. 31B shows cytotoxicity studies using MDA MB231 TNBC cell line transfected using lipofectamine with miRRA 3-p vs. scrambled RNA negative control (NC) (N=4) in the presence of cisplatin*. Transfection efficiency was 93%.

FIG. 31C shows cytotoxicity studies using JL BTL-12 luminal type breast cancer (BC) cell line transfected using lipofectamine with miRRA 3-p vs. scrambled RNA NC (N=3) in the presence of cisplatin*. Transfection efficiency was 87%.

*FIGS. 31A-C: Time zero reflects cell viability post-transfection with miRRA, but before cisplatin treatment. 15 nM miRRA was administered at all doses of cisplatin. Transfection efficiency was 93%.

MiRRA increases the cytotoxicity of cisplatin in a dose-responsive way in three cell lines, MCF7 (stage IV post-hormonal therapy), MDA MB231 (stage IV, post-chemotherapy) and JL BTL-12 (stage III, treatment-naive). The fact that there is an impact in all 3 types of late-stage cell line (post hormonal luminal BC, post chemotherapy TNBC and treatment naïve stage III BC) indicates that miRRA could be effective in all late-stage BCs regardless of whether they went through the bottleneck of treatment.

### Experimental Approach for In Vivo Mouse Model

In order to determine whether miRRA shows in vivo efficacy against triple-negative breast (TNBC) and luminal type breast cancer (BC) cell lines at advanced stages, human tumors need to be created in immunocompromised mice and treated with miRRA (to lower drug resistances via lowering DNA repair) followed by application of Adriamycin or cisplatin. In addition, miRRA by itself will be tested to determine if it has an impact on its own.

Methods: Using a xenograft mouse model to determine the effects of miRRA in combination with Adriamycin/cisplatin. Luminal type cell lines will be grown in the presence of added estradiol in mice. Female NOD/SCID mice will be injected with a BC cell line into the mammary fat pad (1-3×10⁶ cells) and allowed to grow, approximately 5 days to a size no more than 20 mm³ as established by IACUC. Mice will be randomly assigned to the treatment groups: Mock treated (jet-PEI) control (n=5), miRRA+2.5 mg/kg Adriamycin (n=5), miRRA alone (n=5) and drug Adriamycin alone (n=5). Based upon the literature (36-38), microRNAs in animal studies are best delivered in mice to the tumor either via IV (intravenous) with mammary gland tumors or IP (intraperitoneal) injection and flank tumors using the in vivo jet-PEI transfection agent (Polyplus Transfection), once the tumor has been established in the mice. Based on NER and cytotoxicity results found in vitro on the final cell lines which miRRA strand to use in vivo with that cell line will be chosen. A complex of 100 ug of miRRA with 12-16 ul of jet-PEI (NP nitrogen residues per nucleic acid phosphate ratio of 6-8) will be made. Adriamycin/cisplatin will be administered 3 doses a week for 3-4 weeks. miRRA transfection complex will be administered 12-24 hours before each dose at a dose per injection of 10 ug. Tumor size will be measured every 3 days to determine the effects of miRRA and Adriamycin on tumor growth. Animal weight will also be recorded as a measure of general health. Animals will be euthanized, dissected and the tumors viably preserved.

Expected outcomes: End labeled (red spectrum fluorescent tag) miRRA will show tumor uptake. Based on the most recent literature, it is anticipated that IV introduction of miRRA will be successful with mammary gland location of the tumor cells and that it will not be necessary to perform IP injection of drugs in order to see results. However, flank injections of tumor cells with IP introduction of drugs can be performed if this approach becomes necessary.

Data Analysis: Tumor size and weight will be analyzed using IBM SPSS, version 24 (Armonk, N.Y.). One-way analysis of variance (ANOVA) followed by an unpaired Student's t-test will be used to determine if there are any statistical difference between the means of the treatment groups (p≤0.05). In addition, standard regression models will be used to determine whether there are significant differences in the temporal trends between the treatment groups.

### Detailed Procedures: Vertebrate Animals

In the proposed study, 150 female nonobese diabetic/severe combined immunodeficiency (NOD/SCID) mice, aged 8-10 weeks and obtained from Jackson Laboratory will be used. In previous work, cell lines (JL BTL 12) were grown in NOD/SCID mice. Mice will be housed in the state-of-the-art Vivarium at the Center for Collaborative Research at Nova Southeastern University—Davie Campus.

Tumor Induction: Tumors will be established in NOD/SCID mice to investigate the in vivo efficacy of a microRNA alone and in combination with Adriamycin or cisplatin. Tumor cells (1-3×10⁶ cells) will be injected with Matrigel subcutaneously in the mammary fat pad of each mouse After about 5 days, tumor size should be approximately 20 mm³ and the mice will be randomized into different treatment groups: mock treated (jet-PEI) control (n=5), miRRA+2.5 mg/kg Adriamycin (n=5), miRRA alone (n=5) and drug adriamycin alone (n=5). The transfection complex will consist of 100 ug of miRRA with 12-16 ul of jet-PEI (NP nitrogen residues per nucleic acid phosphate ratio of 6-8) according to manufacturer's protocol. MiRRA jet-PEI complex will be administered via IV 24 hours before each dose of chemotherapy agent (Adriamycin/cisplatin) at a dose per injection of 10 ug. MiRRA and the corresponding chemotherapy agent will be administered 3× a week for 3 weeks to determine the effects of miRRA and chemotherapy on tumor growth. If miRRA does not accumulate in the mammary fat pad tumors, then flank injections of tumor cells and IP injection of the miRRA complex will be performed. However, if (and when) the tumor grows and the animal requires pain medication, buprenorphine 0.1-0.3 mg/kg IM or SQ q8-12 hours will be provided as needed as the recommended dosage by the attending veterinarian.

### Experimental Timeline:

Day 0—Tumor induction
Day 5—tumor growth approx. 5 mm³
Day 6-first dose of miRRA jet-PEI complex
Day 7-first dose of Adriamycin/cisplatin
Day 9—tumor measurement and second dose of miRRA jet-PEI
complex
Day 10-second dose of Adriamycin/cisplatin
Day 26—euthanasia and tissue harvest
Euthanasia: The clinical signs that will be used to determine whether an animal will be removed from the study will include weight loss, inability to reach food or water, skin ulceration at injection site and decreased physical activity. Study animals will be monitored 3 days a week for any adverse reactions, as well as measuring tumor size and the Body Conditioning Scoring to determine if the animals need to be humanely euthanized. Body Condition Scoring is a common technique used to evaluate the health of mice and rats in tumor studies. In brief, animals are scored according to the scales based on several categories describing the condition of the animals' fur, eyes, mucous membranes, fat deposition, muscle mass, and behavior. BCS-2 mice show these prominent features: segmentation of vertebral column, thin flesh over dorsal pelvis, little subcutaneous fat, and thein flesh over caudal vertebrae. Detailed descriptions of these scales can be reviewed ad libitum using many references including: Paster, et al. (2009) Endpoints for Mouse Abdominal Tumor Models: Refinement of Current Criteria. Comp. Med. 59(3), 234-241; or Ullman-Cullere and Foltz. (1999). Body Conditioning Scoring: A Rapid and Accurate Method for Assessing Health Status in Mice. Laboratory Animal Science. 49(3), 319-323. If the mice exhibit decreased physical activity that is persistent for greater than 24 hours, then they will be euthanized. If animals exhibit combinations of any of the above abnormalities described above, then they will be euthanized my research staff. Euthanasia will occur if the tumor size reaches 200 mm³ to minimize pain, the mice will be observed 3 days a week to monitor tumor growth. Mice will be humanely euthanized with CO2 followed by cervical dislocation. Carbon dioxide inhalation via SMARTBOX followed by cervical dislocation under isoflurane anesthesia may be performed if it is deemed necessary that the lungs be imaged postmortem. This is to prevent the adverse effects, to the lungs, from using CO2.

### CONCLUSION

The invention described and exemplified herein provides new insights into nucleotide excision repair (NER) regulation in breast cancer (BC) and provides novel treatment strategies, i.e., intravenous miR-145 (miRRA), to effectively treat aggressive breast tumors, particularly those tumors that reoccur. Reduced NER capacity in these tumor cells enables use of genotoxic chemotherapy with much greater efficacy, particularly in the stage III or stage IV scenario.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The compositions and methods described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

### REFERENCE LIST: EXPERIMENTAL PART TWO

1. Li, X., Lewis, M. T., Huang, J., Gutierrez, C., Osborne, C. K., Wu, M. F., Hilsenbeck, S. G., Pavlick, A., Zhang, X., Chamness, G. C., Wong, H., Rosen, J., & Chang, J. C. (2008). Intrinsic resistance of tumorigenic BC cells to chemotherapy. J Natl Cancer Inst, 100(9), 672-679. doi.org/10.1093/inci/djn123.
2. Longley, D. B., & Johnston, P. G. (2005). Molecular mechanisms of drug resistance. J Pathol, 205(2), 275-292. https://doi.org/10.1002/path.1706
3. Latimer J J, Johnson J M, Kelly C M, Miles T D, Beaudry-Rodgers K A, Lalanne N A, Vogel V G, Kanbour-Shakir A, Kelley J L, Johnson R J, Grant S G. (2010) Nucleotide excision repair deficiency is intrinsic in sporadic stage I breast cancer. Proc Natl Acad Sci USA 50: 21725-30. doi:10.1073/pnas.0914772107.
4. Li Q, Damish A W, Frazier Z, Liu D, Reznichenko E, Kamburov A, Bell A, Zhao H, Jordan E J, Gao S P, Ma J, Abbosh P H, Bellmunt J, Plimack E R, Lazaro J-B, Solit D B, Bajorin D, Rosenberg J E, D'Andrea A D, Riaz N, Van Allen E M, Iyer G, Mouw K W. (2018) ERCC2 helicase domain mutations confer nucleotide excision repair deficiency and drive cisplatin sensitivity in muscle-invasive bladder cancer. Clin. Cancer Res. 25: 977-88. doi:10.1158/1078-0432.
5. Koberle B, Masters J R, Hartley J A, Wood R D. (1999) Defective repair of cisplatininduced DNA damage caused by reduced XPA protein in testicular germ cell tumours. Curr. Biol. 9: 273-6. doi:10.1016/s0960-9822(99)80118-3.
6. Latimer J J. (2000). U.S. Pat. No. 6,074,874 Epithelial cell cultures useful for in vitro testing (medium).
7. Latimer J J. Johnson J M, Miles T D, Dimsdale J M, Edwards R P, Kelley J L, Grant S G. (2008) Cell-type-specific level of DNA nucleotide excision repair in primary human mammary and ovarian epithelial cell cultures. Cell Tissue Res 333: 461-7.
8. Visus C, Ito D, Dhir R, Szczepanski M J, Chang Y J, Latimer J J, Grant S G, DeLeo A B. (2011) Identification of hydroxysteroid (17) dehydrogenase type 12 (HSD17B12) as a CD8+ T-cell-defined human tumor antigen of human carcinomas. Cancer Immunol. Immunother. 60: 919-29. doi:10.1007/s00262-011-1001-y.
9. Sajithlal G S, Rothermund K, Zhang F, Dabbs D J, Latimer J J, Grant S G, Prochownik E V. (2010) Permanently blocked stem cells derived from breast cancer cell lines. Stem Cells 28: 1008-18. doi:10.1002/stem.424.
10. Wend P, Runke S, Wend K, Anchondo B, Yesayan M, Jardan M, Hardie N, Loddenkemper C, Ulasov I, Lesniak M S, Wolsky R, Bentolila L A, Grant S G, Elashoof D, Lehr S, Latimer J J, Bose S, Sattar H, Krum S A, Miranda-Carboni G A. (2013) WNT10B/beta-catenin signaling induces HMGA2 and proliferation in metastatic triple-negative breast cancer. EMBO Mol. Med. 5: 1-16. doi:10.1002/emmm.201201320.
11. Matta J, Morales L, Ortiz C, Adams D, Vargas W, Casbas P, Dutil J, Echenique M, Suarez E. (2016) Estrogen receptor expression is associated with DNA repair capacity in breast cancer. PLOS One 11: 1-14. doi: 10.1371/journal.pone.0152422.
12. Ibrahim O, As Sobeai H, Grant S G, Latimer J J. (2018). Nucleotide excision repair is a predictor of early relapse in acute lymphoblastic leukemia. BMC Med. Genomics 11: 95. doi: 10.1186/s12920-018-0422-2.
13. Edge S B, Compton C C. (2010) The American Joint Committee on Cancer: the 7th edition of the AJCC cancer staging manual and the future of TNM. Ann. Surg. Oncol. 17: 14711474. doi: 10.1245/s10434-010-0985-4.
14. Howlader N, Noone A M, Krapcho M, Miller D, Bishop K, Altekruse S F, Kosary C L, Yu M, Ruhl J, Tatalovich Mariotto A,

What is claimed is:

1. A composition for treatment of cancer comprising microRNA.

2. The composition according to claim 1, wherein the microRNA is miR-145.

3. The composition according to claim 2, wherein miR-145 includes a guide strand, a passenger strand, or both guide and passenger strands.

4. The composition according to claim 3, wherein the guide strand is miR-145-5p and the passenger strand is miR-145-3p.

5. The composition according to claim 2, wherein the cancer is at least one of a solid cancer, a late-stage cancer, or a drug-resistant cancer.

6. A pharmaceutical composition for treatment of cancer comprising a therapeutically effective intravenous dosage of microRNA in a liquid pharmaceutical carrier.

7. The composition according to claim 6, wherein the microRNA is miR-145.

8. The composition according to claim 7, wherein miR-145 includes a guide strand, a passenger strand, or both guide and passenger strands.

9. The composition according to claim 8, wherein the guide strand is miR-145-5p and the passenger strand is miR-145-3p.

10. The composition according to claim 7, wherein the cancer is at least one of a solid cancer, a late-stage cancer, or a drug-resistant cancer.

11. A composition for treatment of breast cancer comprising microRNA.

12. The composition according to claim 11, wherein the microRNA is miR-145.

13. The composition according to claim 12, wherein miR-145 includes a guide strand, a passenger strand, or both guide and passenger strands.

14. The composition according to claim 13, wherein the guide strand is miR-145-5p and the passenger strand is miR-145-3p.

15. The composition according to claim 12, wherein the breast cancer is at least one of a late-stage breast cancer, a drug-resistant breast cancer, a triple negative breast cancer (TNBC), or a luminal-type breast cancer.

16. A pharmaceutical composition for treatment of breast cancer comprising a therapeutically effective intravenous dosage of microRNA in a liquid pharmaceutical carrier.

17. The composition according to claim 16, wherein the microRNA is miR-145.

18. The composition according to claim 17, wherein miR-145 includes a guide strand, a passenger strand, or both guide and passenger strands.

19. The composition according to claim 18, wherein the guide strand is miR-145-5p and the passenger strand is miR-145-3p.

20. The composition according to claim 17, wherein the breast cancer is at least one of a late-stage breast cancer, a drug-resistant breast cancer, a triple negative breast cancer (TNBC), or a luminal-type breast cancer.

21. A method for regulating nucleotide excision repair (NER) function in malignant cells, the method comprising:
providing a composition including microRNA; and
administering the composition to the malignant cells.

22. A method for suppressing nucleotide excision repair (NER) function in malignant cells, the method comprising:
providing a composition including microRNA; and
administering the composition to the malignant cells.

23. A method for inhibiting expression of at least one of XPC, XPA, and RPA3 genes in malignant cells, the method comprising:
providing a composition including microRNA; and
administering the composition to the malignant cells.

24. A method for inhibiting expression of at least one of XPC, XPA, and RPA3 protein in malignant cells, the method comprising:
providing a composition including microRNA; and
administering the composition to the malignant cells.

25. A method for increasing sensitivity to DNA-damaging drugs in malignant cells, the method comprising:
providing a composition including microRNA; and
administering the composition to the malignant cells.

26. A method for treating cancer in a subject in need thereof by suppressing nucleotide excision repair (NER) function in cancer cells, the method comprising:
providing a composition including microRNA; and
administering the composition to the cancer cells.

27. The method according to claim 26, further comprising administering a DNA-damaging drug to the subject after administering the composition or concurrently with the composition.

28. The method according to claim 27, wherein administering the DNA-damaging drug includes administering at least one of cisplatin or Adriamycin to the subject.

29. A method for treating a solid cancer, a late-stage cancer, or a drug-resistant cancer in a subject in need thereof by inhibiting expression of at least one of XPC and XPA proteins in cancer cells, the method comprising:
providing a composition including a therapeutically effective intravenous dosage of microRNA in a liquid pharmaceutical carrier; and
administering the composition to the subject, whereby expression of the at least one of XPC and XPA proteins is inhibited in the cancer cells.

30. The method according to claim 29, wherein the providing includes providing a composition including a therapeutically effective injectable dosage of miR-145.

31. The method according to claim 29, wherein the providing includes providing a composition including a therapeutically effective intravenous dosage of guide strand miR-145-5p.

32. The method according to claim 29, further comprising administering a DNA-damaging drug to the subject after administering the composition or concurrently with the composition.

33. The method according to claim 32, wherein administering the DNA-damaging drug includes administering at least one of cisplatin or Adriamycin to the subject.

34. A method for treating breast cancer in a subject in need thereof by suppressing nucleotide excision repair (NER) function in breast cancer cells, the method comprising:
providing a composition including microRNA; and
administering the composition to the breast cancer cells.

35. The method according to claim 34, further comprising administering a DNA-damaging drug to the subject after administering the composition or concurrently with the composition.

36. The method according to claim 35, wherein administering the DNA-damaging drug includes administering at least one of cisplatin or Adriamycin to the subject.

37. A method for treating late-stage breast cancer, drug-resistant breast cancer, triple negative breast cancer (TNBC), or luminal-type breast cancer in a subject in need thereof by inhibiting expression of at least one of XPC and XPA proteins in breast cancer cells, the method comprising:
providing a composition including a therapeutically effective intravenous dosage of microRNA in a liquid pharmaceutical carrier; and
administering the composition to the subject, whereby expression of the at least one of XPC and XPA proteins is inhibited in the breast cancer cells.

38. The method according to claim 37, wherein the providing includes providing a composition including a therapeutically effective injectable dosage of miR-145.

39. The method according to claim 37, wherein the providing includes providing a composition including a therapeutically effective intravenous dosage of guide strand miR-145-5p.

40. The method according to claim 38, further comprising administering a DNA-damaging drug to the subject after administering the composition or concurrently with the composition.

41. The method according to claim 40, wherein administering the DNA-damaging drug includes administering at least one of cisplatin or Adriamycin to the subject.

42. A method for treating late-stage breast cancer, drug-resistant breast cancer, triple negative breast cancer (TNBC), or luminal-type breast cancer in a subject in need thereof by inhibiting expression of RPA3 protein in cancer cells, the method comprising:
providing a composition including a therapeutically effective intravenous dosage of microRNA in a liquid pharmaceutical carrier; and
administering the composition to the subject, whereby expression of the RPA3 protein is inhibited in the cancer cells.

43. The method according to claim 42, wherein the providing includes providing a composition including a therapeutically effective intravenous dosage of miR-145.

44. The method according to claim 42, wherein the providing includes providing a composition including a therapeutically effective injectable dosage of passenger strand miR-145-3p.

45. The method according to claim 42, further comprising administering a DNA-damaging drug to the subject after administering the composition or concurrently with the composition.

46. The method according to claim 45, wherein administering the DNA-damaging drug includes administering at least one of cisplatin or Adriamycin to the subject.

47. A method for treating late-stage breast cancer, drug-resistant breast cancer, triple negative breast cancer (TNBC), or luminal-type breast cancer in a subject in need thereof by inhibiting expression of RPA3 protein in breast cancer cells, the method comprising:
providing a composition including a therapeutically effective intravenous dosage of microRNA in a liquid pharmaceutical carrier; and
administering the composition to the subject, whereby expression of the RPA3 protein is inhibited in the breast cancer cells.

48. The method according to claim 47, wherein the providing includes providing a composition including a therapeutically effective intravenous dosage of miR-145.

49. The method according to claim 47, wherein the providing includes providing a composition including a therapeutically effective injectable dosage of passenger strand miR-145-3p.

50. The method according to claim 47, further comprising administering a DNA-damaging drug to the subject after administering the composition or concurrently with the composition.

51. The method according to claim 50, wherein administering the DNA-damaging drug includes administering at least one of cisplatin or Adriamycin to the subject.

52. A microRNA and a liquid pharmaceutical carrier for use in manufacture of a composition for treating cancer.

53. Use according to claim 52, wherein the microRNA is miR-145.

54. Use according to claim 53, wherein miR-145 includes a guide strand, a passenger strand, or both guide and passenger strands.

55. Use according to claim 54, wherein the guide strand is miR-145-5p and the passenger strand is miR-145-3p.

56. Use according to claim 53, wherein the cancer is at least one of a solid cancer, a breast cancer, a late-stage cancer, or a drug-resistant cancer.

57. Use according to claim 56, wherein the breast cancer is a triple negative breast cancer (TNBC) or a luminal-type breast cancer.

58. Use according to claim 53, further including a DNA-damaging drug.

59. A microRNA and a liquid pharmaceutical carrier for use in manufacture of a composition for regulating nucleotide excision repair (NER) function in malignant cells.

60. Use according to claim 59, wherein the microRNA is miR-145.

61. Use according to claim 59, further including a DNA-damaging drug.

62. A microRNA and a liquid pharmaceutical carrier for use in manufacture of a composition for suppressing nucleotide excision repair (NER) function in malignant cells.

63. Use according to claim 62, wherein the microRNA is miR-145.

64. Use according to claim 62, further including a DNA-damaging drug.

65. A microRNA and a liquid pharmaceutical carrier for use in manufacture of a composition for inhibiting expression of at least one of XPC, XPA, and RPA3 genes in malignant cells.

66. Use according to claim 65, wherein the microRNA is miR-145.

67. Use according to claim 66, further including a DNA-damaging drug.

68. A microRNA and a liquid pharmaceutical carrier for use in manufacture of a composition for inhibiting expression of at least one of XPC, XPA, and RPA3 protein in malignant cells.

69. Use according to claim 68, wherein the microRNA is miR-145.

70. Use according to claim 68, further including a DNA-damaging drug.

71. A microRNA and a liquid pharmaceutical carrier for use in manufacture of a composition for increasing sensitivity to DNA-damaging drugs in malignant cells.

72. Use according to claim 71, wherein the microRNA is miR-145.

73. Use according to claim 71, further including a DNA-damaging drug.
